(19) United States
(12) Patent Application Publication
(10) Pub. No.: US 2011/0288122 A1
(43) Pub. Date: Nov. 24, 2011

(54) Pharmaceutical Compositions and Administrations Thereof

(75) Inventors: Fredrick F. Van Goor, San Diego, CA (US); William Lawrence Burton, San Diego, CA (US); Chien-Jung Huang, Encinitas, CA (US); Paul Adrian Negulescu, Del Mar, CA (US); Haihui Yu, San Diego, CA (US)

(73) Assignee: Vertex Pharmaceuticals Incorporated, Cambridge, MA (US)

(21) Appl. No.: 13/112,982

(22) Filed: May 20, 2011

Related U.S. Application Data
(60) Provisional application No. 61/346,798, filed on May 20, 2010.

(51) Int. Cl. A61K 31/47; A61P 11/06; A61P 11/08; A61P 11/02; A61P 1/00; A61P 1/18; A61P 1/16; A61P 7/00; A61P 7/02; A61P 3/00; A61P 43/00; A61P 3/10; A61P 35/00; A61P 19/08; A61P 25/28; A61P 1/02; A61P 25/16; A61P 25/00; A61P 21/00; A61P 27/02; A61P 19/10; A61P 25/08; A61P 11/00

(52) U.S. Cl. 514/312

(57) ABSTRACT

The present invention relates to the use of N-[2,4-bis(1,1-dimethylethyl)-5-hydroxyphenyl]-1,4-dihydro-4-oxoquinoline-3-carboxamide, solids forms, and pharmaceutical compositions thereof for the treatment of CFTR mediated diseases, particularly cystic fibrosis, in patients possessing specific genetic mutations.

### PRIORITY CLAIM

This application claims priority to U.S. Provisional application Ser. No. 61/346,798, filed on May 20, 2010. The entire contents of this priority document is incorporated by reference herein.

### FIELD OF THE INVENTION

The present invention relates to the use of N-[2,4-bis(1,1-dimethylethyl)-5-hydroxyphenyl]-1,4-dihydro-4-oxoquinoline-3-carboxamide, solids forms, and pharmaceutical compositions thereof for the treatment of CFTR mediated diseases, particularly cystic fibrosis, in patients possessing specific genetic mutations.

### BACKGROUND

Cystic fibrosis (CF) is a recessive genetic disease that affects approximately 30,000 children and adults in the United States and approximately 30,000 children and adults in Europe. Despite progress in the treatment of CF, there is no cure.

CF is caused by mutations in the cystic fibrosis transmembrane conductance regulator (CFTR) gene that encodes an epithelial chloride ion channel responsible for aiding in the regulation of salt and water absorption and secretion in various tissues. Small molecule drugs, known as potentiators that increase the probability of CFTR channel opening, represent one potential therapeutic strategy to treat CF. Potentiators of this type are disclosed in WO 2006/002421, which is herein incorporated by reference in its entirety. Another potential therapeutic strategy involves small molecule drugs known as CF correctors that increase the number and function of CFTR channels. Correctors of this type are disclosed in WO 2007/117715, which is herein incorporated by reference in its entirety.

Specifically, CFTR is a cAMP/ATP-mediated anion channel that is expressed in a variety of cells types, including absorptive and secretory epithelia cells, where it regulates anion flux across the membrane, as well as the activity of other ion channels and proteins. In epithelia cells, normal functioning of CFTR is critical for the maintenance of electrolyte transport throughout the body, including respiratory and digestive tissue. CFTR is composed of approximately 1480 amino acids that encode a protein made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). A defect in this gene causes mutations in CFTR resulting in cystic fibrosis (“CF”), the most common fatal genetic disease in humans. Cystic fibrosis affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease.

In patients with CF, mutations in CFTR endogenously expressed in respiratory epithelia leads to reduced apical anion secretion causing an imbalance in ion and fluid transport. The resulting decrease in anion transport contributes to enhanced mucus accumulation in the lung and the accompanying microbial infections that ultimately cause death in CF patients. In addition to respiratory disease, CF patients typically suffer from gastrointestinal problems and pancreatic insufficiency that, if left untreated, results in death. In addition, the majority of males with cystic fibrosis are infertile and fertility is decreased among females with cystic fibrosis. In contrast to the severe effects of two copies of the CF associated gene, individuals with a single copy of the CF associated gene exhibit increased resistance to cholera and to dehydration resulting from diarrhea—perhaps explaining the relatively high frequency of the CF gene within the population.

Sequence analysis of the CFTR gene of CF chromosomes has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). The most prevalent mutation is a deletion of phenylalanine at position 508 of the CFTR amino acid sequence, and is commonly referred to as ΔF508-CFTR. This mutation occurs in approximately 70% of the cases of cystic fibrosis and is associated with a severe disease.

The deletion of residue 508 in ΔF508-CFTR prevents the nascent protein from folding correctly. This results in the inability of the mutant protein to exit the ER, and traffic to the plasma membrane. As a result, the number of channels present in the membrane is far less than observed in cells expressing wild-type CFTR. In addition to impaired trafficking, the mutation results in defective channel gating. Together, the reduced number of channels in the membrane and the defective gating lead to reduced anion transport across epithelia leading to defective ion and fluid transport. (Quinton, P. M. (1990), FASEB J. 4: 2709-2727). Studies have shown, however, that the reduced numbers of ΔF508-CFTR in the membrane are functional, albeit less than wild-type CFTR. (Dalemans et al. (1991), Nature Lond. 354: 526-528; Denning et al., supra; Pasyk and Foskett (1995), J. Cell. Biochem. 270: 12347-50). In addition to ΔF508-CFTR, other disease causing mutations in CFTR that result in defective trafficking, synthesis, and/or channel gating could be up- or down-regulated to alter anion secretion and modify disease progression and/or severity.

Although CFTR transports a variety of molecules in addition to anions, it is clear that this role (the transport of anions) represents one element in an important mechanism of transporting ions and water across the epithelium. The other elements include the epithelial Na⁺ channel, ENaC, Na⁺/2Cl⁻/K⁺ co-transporter, Na⁺—K⁺-ATPase pump and the basolateral membrane K⁺ channels, that are responsible for the uptake of chloride into the cell.

These elements work together to achieve directional transport across the epithelium via their selective expression and localization within the cell. Chloride absorption takes place by the coordinated activity of ENaC and CFTR present on the apical membrane and the Na⁺—K⁺-ATPase pump and Cl⁻ ion channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

As discussed above, it is believed that the deletion of residue 508 in ΔF508-CFTR prevents the nascent protein from folding correctly, resulting in the inability of this mutant protein to exit the ER, and traffic to the plasma membrane. As a result, insufficient amounts of the mature protein are present at the plasma membrane and chloride transport within epithelial tissues is significantly reduced. In fact, this cellular phenomenon of defective ER processing of ABC transporters by the ER machinery has been shown to be the underlying basis not only for CF disease, but for a wide range of other isolated and inherited diseases.

Accordingly, there is a need for novel treatments of CFTR mediated diseases.

### SUMMARY

These and other needs are met by the present invention which is directed to method of treating CFTR comprising administering with N-[2,4-bis(1,1-dimethylethyl)-5-hydroxyphenyl]-1,4-dihydro-4-oxoquinoline-3-carboxamide (Compound 1) to a patient possessing a human CFTR mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G.

In one aspect, the invention provides a method of treating CFTR comprising administering Compound 1 to a patient possessing a human CFTR mutation selected from G178R, G551S, G970R, G1244E, S1255P, G1349D, S549N, S549R, S1251N, E193K, F1052V and G1069R. In one embodiment of this aspect, the invention provides a method of treating CFTR comprising administering Compound 1 to a patient possessing a human CFTR mutation selected from G178R, G551S, G970R, G1244E, S1255P, G1349D, S549N, S549R and S1251N. In another embodiment of this aspect, the invention provides a method of treating CFTR comprising administering Compound 1 to a patient possessing a human CFTR mutation selected from E193K, F1052V and G1069R. In some embodiments of this aspect, the method produces a greater than 10-fold increase in chloride transport relative to baseline chloride transport.

In another aspect, the invention provides a method of treating CFTR comprising administering Compound 1 to a patient possessing a human CFTR mutation selected from R117C, D110H, R347H, R352Q, E56K, P67L, L206W, A455E, D579G, S1235R, S945L, R1070W, F1074L, D110E, D1270N and D1152H. In one embodiment of this aspect, the method produces an increase in chloride transport which is greater or equal to 10% above the baseline chloride transport.

In another aspect, the invention provides a method of treating CFTR comprising administering Compound 1 to a patient possessing a human CFTR mutation selected from 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G. In one embodiment of this aspect, the method comprises administering Compound 1 to a patient possessing a human CFTR mutation selected from 1717−1G->A, 1811+1.6 kbA->G, 2789+5G->A, 3272−26A->G and 3849+10 kbC->T. In still another embodiment of this aspect, the method comprises administering Compound 1 to a patient possessing a human CFTR mutation selected from 2789+5G->A and 3272−26A->G.

In one aspect, the invention provides a method of treating CFTR comprising administering Compound 1 to a patient possessing a human CFTR mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G, and a human CFTR mutation selected from ΔF508, R117H, and G551D.

In one aspect, the invention provides a method of treating CFTR comprising administering Compound 1 to a patient possessing a human CFTR mutation selected from G178R, G551S, G970R, G1244E, S1255P, G1349D, S549N, S549R, S1251N, E193K, F1052V and G1069R, and a human CFTR mutation selected from ΔF508, R117H, and G551D. In one embodiment of this aspect, the invention provides a method of treating CFTR comprising administering Compound 1 to a patient possessing a human CFTR mutation selected from G178R, G551S, G970R, G1244E, S1255P, G1349D, S549N, S549R and S1251N, and a human CFTR mutation selected from ΔF508, R117H, and G551D. In another embodiment of this aspect, the invention provides a method of treating CFTR comprising administering Compound 1 to a patient possessing a human CFTR mutation selected from E193K, F1052V and G1069R, and a human CFTR mutation selected from ΔF508, R117H, and G551D. In some embodiments of this aspect, the method produces a greater than 10-fold increase in chloride transport relative to baseline chloride transport.

In another aspect, the invention provides a method of treating CFTR comprising administering Compound 1 to a patient possessing a human CFTR mutation selected from R117C, D110H, R347H, R352Q, E56K, P67L, L206W, A455E, D579G, S1235R, S945L, R1070W, F1074L, D110E, D1270N and D1152H, and a human CFTR mutation selected from ΔF508, R117H, and G551D. In one embodiment of this aspect, the method produces an increase in chloride transport which is greater or equal to 10% above the baseline chloride transport.

In another aspect, the invention provides a method of treating CFTR comprising administering Compound 1 to a patient possessing a human CFTR mutation selected from 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G, and a human CFTR mutation selected from ΔF508, R117H, and G551D. In one embodiment of this aspect, the method comprises administering Compound 1 to a patient possessing a human CFTR mutation selected from 1717−1G->A, 1811+1.6 kbA->G, 2789+5G->A, 3272−26A->G and 3849+10 kbC->T, and a human CFTR mutation selected from ΔF508, R117H, and G551D. In still another embodiment of this aspect, the method comprises administering Compound 1 to a patient possessing a human CFTR mutation selected from 2789+5G->A and 3272−26A->G, and a human CFTR mutation selected from ΔF508, R117H.

In one aspect, the invention provides a method of treating CFTR comprising administering Compound 1 to a patient possessing one or more human CFTR mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G, and a human CFTR mutation selected from ΔF508, R117H, and G551D.

In one aspect, the invention provides a method of treating CFTR comprising administering Compound 1 to a patient possessing one or more human CFTR mutations selected from G178R, G551S, G970R, G1244E, S1255P, G1349D, S549N, S549R, S1251N, E193K, F1052V and G1069R. In one embodiment of this aspect, the invention provides a method of treating CFTR comprising administering Compound 1 to a patient possessing one or more human CFTR mutations selected from G178R, G551S, G970R, G1244E, S1255P, G1349D, S549N, S549R and S1251N. In another embodiment of this aspect, the invention provides a method of treating CFTR comprising administering Compound 1 to a patient possessing one or more human CFTR mutations selected from E193K, F1052V and G1069R. In some embodiments of this aspect, the method produces a greater than 10-fold increase in chloride transport relative to baseline chloride transport.

In another aspect, the invention provides a method of treating CFTR comprising administering Compound 1 to a patient possessing one or more human CFTR mutations selected from R117C, D110H, R347H, R352Q, E56K, P67L, L206W, A455E, D579G, S1235R, S945L, R1070W, F1074L, D110E, D1270N and D1152H. In one embodiment of this aspect, the method produces an increase in chloride transport which is greater or equal to 10% above the baseline chloride transport.

In another aspect, the invention provides a method of treating CFTR comprising administering Compound 1 to a patient possessing one or more human CFTR mutations selected from 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G. In one embodiment of this aspect, the method comprises administering Compound 1 to a patient possessing one or more human CFTR mutations selected from 1717−1G->A, 1811+1.6 kbA->G, 2789+5G->A, 3272−26A->G and 3849+10 kbC->T. In still another embodiment of this aspect, the method comprises administering Compound 1 to a patient possessing one or more human CFTR mutations selected from 2789+5G->A and 3272−26A->G.

In one aspect, the invention provides a method of treating CFTR comprising administering Compound 1 to a patient possessing one or more human CFTR mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G, and a human CFTR mutation selected from ΔF508, R117H, and G551D, and one or more human CFTR mutations selected from ΔF508, R117H, and G551D.

In one aspect, the invention provides a method of treating CFTR comprising administering Compound 1 to a patient possessing one or more human CFTR mutations selected from G178R, G551S, G970R, G1244E, S1255P, G1349D, S549N, S549R, S1251N, E193K, F1052V and G1069R, and one or more human CFTR mutations selected from ΔF508, R117H, and G551D. In one embodiment of this aspect, the invention provides a method of treating CFTR comprising administering Compound 1 to a patient possessing one or more human CFTR mutations selected from G178R, G551S, G970R, G1244E, S1255P, G1349D, S549N, S549R and S1251N, and one or more human CFTR mutations selected from ΔF508, R117H, and G551D. In another embodiment of this aspect, the invention provides a method of treating CFTR comprising administering Compound 1 to a patient possessing one or more human CFTR mutations selected from E193K, F1052V and G1069R, and one or more human CFTR mutations selected from ΔF508, R117H, and G551D. In some embodiments of this aspect, the method produces a greater than 10-fold increase in chloride transport relative to baseline chloride transport.

In another aspect, the invention provides a method of treating CFTR comprising administering Compound 1 to a patient possessing one or more human CFTR mutations selected from R117C, D110H, R347H, R352Q, E56K, P67L, L206W, A455E, D579G, S1235R, S945L, R1070W, F1074L, D110E, D1270N and D1152H, and one or more human CFTR mutations selected from ΔF508, R117H, and G551D. In one embodiment of this aspect, the method produces an increase in chloride transport which is greater or equal to 10% above the baseline chloride transport.

In another aspect, the invention provides a method of treating CFTR comprising administering Compound 1 to a patient possessing one or more human CFTR mutations selected from 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G, and one or more human CFTR mutations selected from ΔF508, R117H, and G551D. In one embodiment of this aspect, the method comprises administering Compound 1 to a patient possessing one or more human CFTR mutations selected from 1717−1G->A, 1811+1.6 kbA->G, 2789+5G->A, 3272−26A->G and 3849+10 kbC->T, and one or more human CFTR mutations selected from ΔF508, R117H, and G551D. In still another embodiment of this aspect, the method comprises administering Compound 1 to a patient possessing one or more human CFTR mutations selected from 2789+5G->A and 3272−26A->G, and one or more human CFTR mutations selected from ΔF508, R117H, and G551D.

In any of the foregoing aspects, the method can include administration of Compound 1, Compound 1 Form C, or any of the formulations of Compound 1 described herein in section IV.

### LIST OF FIGURES

FIG. 1-1 is an exemplary X-Ray powder diffraction pattern of Compound 1 Form C.

FIG. 1-2 is an exemplary DSC trace of Compound 1 Form C.

FIG. 1-3 is an exemplary TGA trace of Compound 1 Form C.

FIG. 1-4 is an exemplary Raman spectrum of Compound 1 Form C.

FIG. 1-5 is an exemplary FTIR spectrum of Compound 1 Form C.

FIG. 1-6 is Solid State NMR Spectrum of Compound 1 Form C.

### DETAILED DESCRIPTION

### I. Definitions

As used herein, the following definitions shall apply unless otherwise indicated.

The term “ABC-transporter” as used herein means an ABC-transporter protein or a fragment thereof comprising at least one binding domain, wherein said protein or fragment thereof is present in vivo or in vitro. The term “binding domain” as used herein means a domain on the ABC-transporter that can bind to a modulator. See, e.g., Hwang, T. C. et al., J. Gen. Physiol. (1998): 111(3), 477-90.

The term “CFTR” as used herein means cystic fibrosis transmembrane conductance regulator.

As used herein, the terms “ΔF508” and “F508del” are used interchangeably.

As used herein, the term “active pharmaceutical ingredient” or “API” refers to a biologically active compound. Exemplary APIs include the CF potentiator N-[2,4-bis(1,1-dimethylethyl)-5-hydroxyphenyl]-1,4-dihydro-4-oxoquinoline-3-carboxamide (Compound 1).

The term “modulating” as used herein means increasing or decreasing by a measurable amount.

The term “normal CFTR” or “normal CFTR function” as used herein means wild-type like CFTR without any impairment due to environmental factors such as smoking, pollution, or anything that produces inflammation in the lungs.

The term “reduced CFTR” or “reduced CFTR function” as used herein means less than normal CFTR or less than normal CFTR function.

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

As used herein, the term “substantially amorphous” refers to a solid material having little or no long range order in the position of its molecules. For example, substantially amorphous materials have less than about 15% crystallinity (e.g., less than about 10% crystallinity or less than about 5% crystallinity). It is also noted that the term ‘substantially amorphous’ includes the descriptor, ‘amorphous’, which refers to materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g. single molecules, colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in crystalline polymer; a crystalline drug in an amorphous polymer; or a crystalline drug in crystalline polymer. In this invention, a solid dispersion can include an amorphous drug in an amorphous polymer or an amorphous drug in crystalline polymer. In some embodiments, a solid dispersion includes the polymer constituting the dispersed phase, and the drug constitutes the continuous phase. Or, a solid dispersion includes the drug constituting the dispersed phase, and the polymer constitutes the continuous phase.

As used herein, the term “solid dispersion” generally refers to a solid dispersion of two or more components, usually one or more drugs (e.g., one drug (e.g., Compound 1)) and polymer, but possibly containing other components such as surfactants or other pharmaceutical excipients, where the drug(s) (e.g., Compound 1) is substantially amorphous (e.g., having about 15% or less (e.g., about 10% or less, or about 5% or less)) of crystalline drug (e.g., N-[2,4-bis(1,1-dimethylethyl)-5-hydroxyphenyl]-1,4-dihydro-4-oxoquinoline-3-carboxamide) or amorphous (i.e., having no crystalline drug), and the physical stability and/or dissolution and/or solubility of the substantially amorphous or amorphous drug is enhanced by the other components. Solid dispersions typically include a compound dispersed in an appropriate carrier medium, such as a solid state carrier. For example, a carrier comprises a polymer (e.g., a water-soluble polymer or a partially water-soluble polymer) and can include optional excipients such as functional excipients (e.g., one or more surfactants) or nonfunctional excipients (e.g., one or more fillers). Another exemplary solid dispersion is a co-precipitate or a co-melt of N-[2,4-bis(1,1-dimethylethyl)-5-hydroxyphenyl]-1,4-dihydro-4-oxoquinoline-3-carboxamide with at least one polymer.

A “Co-precipitate” is a product after dissolving a drug and a polymer in a solvent or solvent mixture followed by the removal of the solvent or solvent mixture. Sometimes the polymer can be suspended in the solvent or solvent mixture. The solvent or solvent mixture includes organic solvents and supercritical fluids. A “co-melt” is a product after heating a drug and a polymer to melt, optionally in the presence of a solvent or solvent mixture, followed by mixing, removal of at least a portion of the solvent if applicable, and cooling to room temperature at a selected rate.

As used herein “crystalline” refers to compounds or compositions where the structural units are arranged in fixed geometric patterns or lattices, so that crystalline solids have rigid long range order. The structural units that constitute the crystal structure can be atoms, molecules, or ions. Crystalline solids show definite melting points.

As used herein the phrase “substantially crystalline”, means a solid material that is arranged in fixed geometric patterns or lattices that have rigid long range order. For example, substantially crystalline materials have more than about 85% crystallinity (e.g., more than about 90% crystallinity or more than about 95% crystallinity). It is also noted that the term ‘substantially crystalline’ includes the descriptor ‘crystalline’, which is defined in the previous paragraph.

As used herein, “crystallinity” refers to the degree of structural order in a solid. For example, Compound 1, which is substantially amorphous, has less than about 15% crystallinity, or its solid state structure is less than about 15% crystalline. In another example, Compound 1, which is amorphous, has zero (0%) crystallinity.

As used herein, an “excipient” is an inactive ingredient in a pharmaceutical composition. Examples of excipients include fillers or diluents, surfactants, binders, glidants, lubricants, disintegrants, and the like.

As used herein, a “disintegrant” is an excipient that hydrates a pharmaceutical composition and aids in tablet dispersion. Examples of disintegrants include sodium croscarmellose and/or sodium starch glycolate.

As used herein, a “diluent” or “filler” is an excipient that adds bulkiness to a pharmaceutical composition. Examples of fillers include lactose, sorbitol, celluloses, calcium phosphates, starches, sugars (e.g., mannitol, sucrose, or the like) or any combination thereof.

As used herein, a “surfactant” is an excipient that imparts pharmaceutical compositions with enhanced solubility and/or wetability. Examples of surfactants include sodium lauryl sulfate (SLS), sodium stearyl fumarate (SSF), polyoxyethylene 20 sorbitan mono-oleate (e.g., Tween™), or any combination thereof.

As used herein, a “binder” is an excipient that imparts a pharmaceutical composition with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts a pharmaceutical compositions with enhanced flow properties. Examples of glidants include colloidal silica and/or talc.

As used herein, a “colorant” is an excipient that imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof.

As used herein, a “lubricant” is an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

As used herein, “friability” refers to the property of a tablet to remain intact and withhold its form despite an external force of pressure. Friability can be quantified using the mathematical expression presented in equation 1:

$$\% friabiliy = 100 \times \frac{( {W}_{0} - {W}_{f} )}{{W}_{0}} \qquad ( 1 )$$

wherein W₀ is the original weight of the tablet and W_{f} is the final weight of the tablet after it is put through the friabilator.

Friability is measured using a standard USP testing apparatus that tumbles experimental tablets for 100 revolutions. Some tablets of the present invention have a friability of less than about 1% (e.g., less than about 0.75%, less than about 0.50%, or less than about 0.30%)

As used herein, “mean particle diameter” is the average particle diameter as measured using techniques such as laser light scattering, image analysis, or sieve analysis.

As used herein, “bulk density” is the mass of particles of material divided by the total volume the particles occupy. The total volume includes particle volume, inter-particle void volume and internal pore volume. Bulk density is not an intrinsic property of a material; it can change depending on how the material is processed.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools, probes in biological assays or as therapeutic agents.

Examples of suitable solvents are, but not limited to, water, methanol, dichloromethane (DCM), acetonitrile, dimethylformamide (DMF), ethyl acetate (EtOAc), isopropyl alcohol (IPA), isopropyl acetate (IPAc), tetrahydrofuran (THF), methyl ethyl ketone (MEK), t-butanol and N-methyl pyrrolidone (NMP).

### II. Compositions

### II.A. Compound 1

Compound 1 is known by the name N-[2,4-bis(1,1-dimethylethyl)-5-hydroxyphenyl]-1,4-dihydro-4-oxoquinoline-3-carboxamide and by the name N-(5-hydroxy-2,4-di-tert-butylphenyl)-4-oxo-1H-quinoline-3-carboxamide.

In one aspect, the invention is directed to a composition comprising Compound 1 for the treatment of CFTR in patients possessing one or more of the CFTR genetic mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−10->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G.

In another aspect, the invention is directed to a composition comprising Compound 1 for the treatment of CFTR in patients possessing one or more of the CFTR genetic mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H.

Compound 1 can be prepared by known methods. An exemplary synthesis of Compound 1 is shown in the examples below and in Schemes 1-4, 1-5, 1-6, and 1-7.

In any of the foregoing aspects, the method can include administration of Compound 1, Compound 1 Form C, or any of the formulations of Compound 1 described herein in section IV.

### II.A.1. EXAMPLES

### Synthesis of Compound 1

### II.A.1.a. Synthesis of Acid Moiety of Compound 1

The synthesis of the acid moiety 4-Oxo-1,4-dihydroquinoline-3-carboxylic acid 26, is summarized in Scheme 1-4.

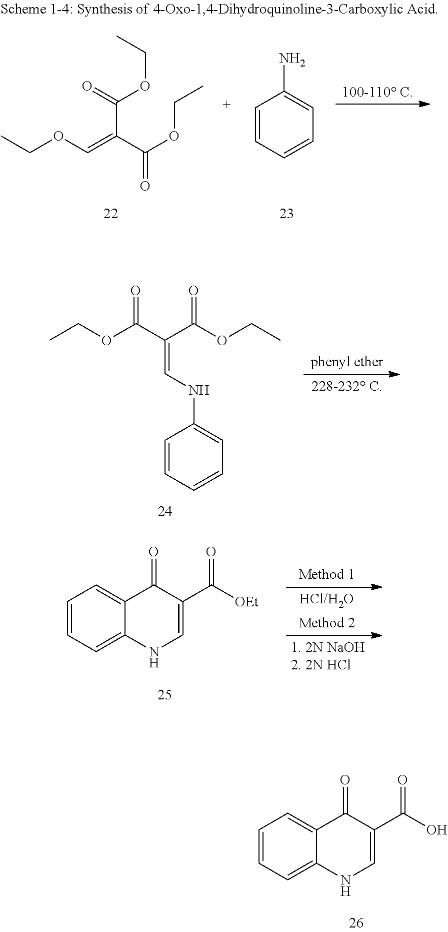

### Example 1a

### Ethyl 4-oxo-1,4-dihydroquinoline-3-carboxylate (25)

Compound 23 (4.77 g, 47.7 mmol) was added dropwise to Compound 22 (10 g, 46.3 mmol) with subsurface N₂ flow to drive out ethanol below 30° C. for 0.5 hours. The solution was then heated to 100-110° C. and stirred for 2.5 hours. After cooling the mixture to below 60° C., diphenyl ether was added. The resulting solution was added dropwise to diphenyl ether that had been heated to 228-232° C. for 1.5 hours with subsurface N₂ flow to drive out ethanol. The mixture was stirred at 228-232° C. for another 2 hours, cooled to below 100° C. and then heptane was added to precipitate the product. The resulting slurry was stirred at 30° C. for 0.5 hours. The solids were then filtered, and the cake was washed with heptane and dried in vacuo to give Compound 25 as a brown solid. ¹H NMR (DMSO-d₆; 400 MHz) δ 12.25 (s), δ 8.49 (d), δ 8.10 (m), δ 7.64 (m), δ 7.55 (m), δ 7.34 (m), δ 4.16 (q), δ 1.23 (t).

### Example 1b

### 4-Oxo-1,4-dihydroquinoline-3-carboxylic acid (26)

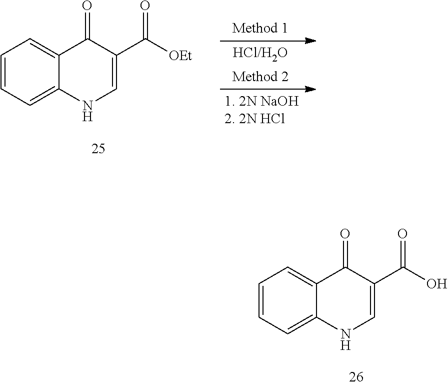

### Method 1

Compound 25 (1.0 eq) was suspended in a solution of HCl (10.0 eq) and H₂O (11.6 vol). The slurry was heated to 85-90° C., although alternative temperatures are also suitable for this hydrolysis step. For example, the hydrolysis can alternatively be performed at a temperature of from about 75 to about 100° C. In some instances, the hydrolysis is performed at a temperature of from about 80 to about 95° C. In others, the hydrolysis step is performed at a temperature of from about 82 to about 93° C. (e.g., from about 82.5 to about 92.5° C. or from about 86 to about 89° C.). After stirring at 85-90° C. for approximately 6.5 hours, the reaction was sampled for reaction completion. Stirring may be performed under any of the temperatures suited for the hydrolysis. The solution was then cooled to 20-25° C. and filtered. The reactor/cake was rinsed with H₂O (2 vol×2). The cake was then washed with 2 vol H₂O until the pH≧3.0. The cake was then dried under vacuum at 60° C. to give Compound 26.

### Method 2

Compound 25 (11.3 g, 52 mmol) was added to a mixture of 10% NaOH (aq) (10 mL) and ethanol (100 mL). The solution was heated to reflux for 16 hours, cooled to 20-25° C. and then the pH was adjusted to 2-3 with 8% HCl. The mixture was then stirred for 0.5 hours and filtered. The cake was washed with water (50 mL) and then dried in vacuo to give Compound 26 as a brown solid. ¹H NMR (DMSO-d₆; 400 MHz) δ 15.33 (s), δ 13.39 (s), δ 8.87 (s), δ 8.26 (m), δ 7.87 (m), δ 7.80 (m), δ 7.56 (m).

### II.A.1.b. Synthesis of Amine Moiety of Compound 1

The synthesis of the amine moiety 32, is summarized in Scheme 1-5.

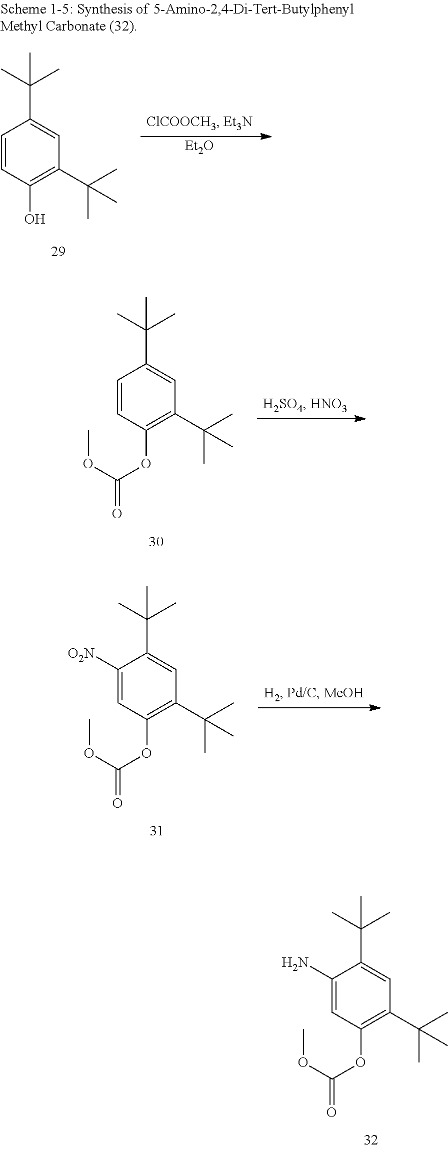

### Example 1c

### 2,4-Di-tert-butylphenyl methyl carbonate (30)

### Method 1

To a solution of 2,4-di-tert-butyl phenol (29) (10 g, 48.5 mmol) in diethyl ether (100 mL) and triethylamine (10.1 mL, 72.8 mmol), was added methyl chloroformate (7.46 mL, 97 mmol) dropwise at 0° C. The mixture was then allowed to warm to room temperature and stir for an additional 2 hours. An additional 5 mL triethylamine and 3.7 mL methyl chloroformate was then added and the reaction stirred overnight. The reaction was then filtered, the filtrate was cooled to 0° C., and an additional 5 mL triethylamine and 3.7 mL methyl chloroformate was then added and the reaction was allowed to warm to room temperature and then stir for an additional 1 hour. At this stage, the reaction was almost complete and was worked up by filtering, then washing with water (2×), followed by brine. The solution was then concentrated to produce a yellow oil and purified using column chromatography to give Compound 30. ¹H NMR (400 MHz, DMSO-d₆) δ 7.35 (d, J=2.4 Hz, 1H), 7.29 (dd, J=8.4, 2.4 Hz, 1H), 7.06 (d, J=8.4 Hz, 1H), 3.85 (s, 3H), 1.30 (s, 9H), 1.29 (s, 9H).

### Method 2

To a reactor vessel charged with 4-dimethylaminopyridine (DMAP, 3.16 g, 25.7 mmol) and 2,4-ditert-butyl phenol (Compound 29, 103.5 g, 501.6 mmol) was added methylene chloride (415 g, 313 mL) and the solution was agitated until all solids dissolved. Triethylamine (76 g, 751 mmol) was then added and the solution was cooled to 0-5° C. Methyl chloroformate (52 g, 550.3 mmol) was then added dropwise over 2.5-4 hours, while keeping the solution temperature between 0-5° C. The reaction mixture was then slowly heated to 23-28° C. and stirred for 20 hours. The reaction was then cooled to 10-15° C. and charged with 150 mL water. The mixture was stirred at 15-20° C. for 35-45 minutes and the aqueous layer was then separated and extracted with 150 mL methylene chloride. The organic layers were combined and neutralized with 2.5% HCl (aq) at a temperature of 5-20° C. to give a final pH of 5-6. The organic layer was then washed with water and concentrated in vacuo at a temperature below 20° C. to 150 mL to give Compound 30.

### Example 1d

### 5-Nitro-2,4-di-tert-butylphenyl methyl carbonate (31)

### Method 1

To a stirred solution of Compound 30 (6.77 g, 25.6 mmol) was added 6 mL of a 1:1 mixture of sulfuric acid and nitric acid at 0° C. dropwise. The mixture was allowed to warm to room temperature and stirred for 1 hour. The product was purified using liquid chromatography (ISCO, 120 g, 0-7% EtOAc/Hexanes, 38 min) producing about an 8:1-10:1 mixture of regioisomers of Compound 31 as a white solid. ¹H NMR (400 MHz, DMSO-d₆) δ 7.63 (s, 1H), 7.56 (s, 1H), 3.87 (s, 3H), 1.36 (s, 9H), 1.32 (s, 9H). HPLC ret. time 3.92 min 10-99% CH₃CN, 5 min run; ESI-MS 310 m/z (MH)⁺.

### Method 2

To Compound 30 (100 g, 378 mmol) was added DCM (540 g, 408 mL). The mixture was stirred until all solids dissolved, and then cooled to −5-0° C. Concentrated sulfuric acid (163 g) was then added dropwise, while maintaining the initial temperature of the reaction, and the mixture was stirred for 4.5 hours. Nitric acid (62 g) was then added dropwise over 2-4 hours while maintaining the initial temperature of the reaction, and was then stirred at this temperature for an additional 4.5 hours. The reaction mixture was then slowly added to cold water, maintaining a temperature below 5° C. The quenched reaction was then heated to 25° C. and the aqueous layer was removed and extracted with methylene chloride. The combined organic layers were washed with water, dried using Na₂SO₄, and concentrated to 124-155 mL. Hexane (48 g) was added and the resulting mixture was again concentrated to 124-155 mL. More hexane (160 g) was subsequently added to the mixture. The mixture was then stirred at 23-27° C. for 15.5 hours, and was then filtered. To the filter cake was added hexane (115 g), the resulting mixture was heated to reflux and stirred for 2-2.5 hours. The mixture was then cooled to 3-7° C., stirred for an additional 1-1.5 hours, and filtered to give Compound 31 as a pale yellow solid.

### Example 1e

### 5-Amino-2,4-di-tert-butylphenyl methyl carbonate (32)

2,4-Di-tert-butyl-5-nitrophenyl methyl carbonate (1.00 eq) was charged to a suitable hydrogenation reactor, followed by 5% Pd/C (2.50 wt % dry basis, Johnson-Matthey Type 37). MeOH (15.0 vol) was charged to the reactor, and the system was closed. The system was purged with N₂ (g), and was then pressurized to 2.0 Bar with H₂ (g). The reaction was performed at a reaction temperature of 25° C.+/−5° C. When complete, the reaction was filtered, and the reactor/cake was washed with MeOH (4.00 vol). The resulting filtrate was distilled under vacuum at no more than 50° C. to 8.00 vol. Water (2.00 vol) was added at 45° C.+/−5° C. The resultant slurry was cooled to 0° C.+/−5. The slurry was held at 0° C.+/−5° C. for no less than 1 hour, and filtered. The cake was washed once with 0° C.+/−5° C. MeOH/H₂O (8:2) (2.00 vol). The cake was dried under vacuum (−0.90 bar and −0.86 bar) at 35° C.-40° C. to give Compound 32. ¹H NMR (400 MHz, DMSO-d₆) δ 7.05 (s, 1H), 6.39 (s, 1H), 4.80 (s, 2H), 3.82 (s, 311), 1.33 (s, 9H), 1.23 (s, 914).

Once the reaction was complete, the resulting mixture was diluted with from about 5 to 10 volumes of MeOH (e.g., from about 6 to about 9 volumes of MeOH, from about 7 to about 8.5 volumes of MeOH, from about 7.5 to about 8 volumes of MeOH, or about 7.7 volumes of MeOH), heated to a temperature of about 35±5° C., and filtered to remove palladium. The reactor cake was washed before combining the filtrate and wash, distilling, adding water, cooling, filtering, washing and drying the product cake as described above.

### II.A.1.c. Synthesis of Compound 1 by Acid and Amine Moiety Coupling

The coupling of the acid moiety to the amine moiety is summarized in Scheme 1-6.

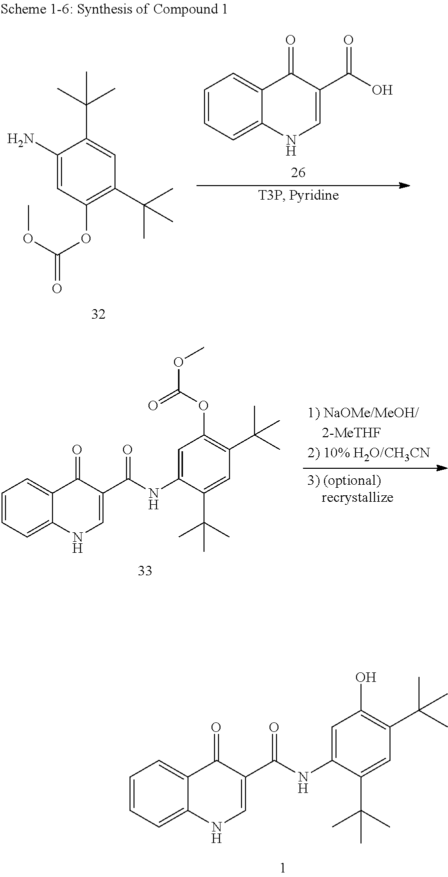

### Example 1f

### N-(2,4-di-tert-butyl-5-hydroxyphenyl)-4-oxo-1,4-dihydroquinoline-3-carboxamide (1)

4-Oxo-1,4-dihydroquinoline-3-carboxylic acid (26) (1.0 eq) and 5-amino-2,4-di-tert-butylphenyl methyl carbonate (32) (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 25.0° C.+/−2.5° C. 2-MeTHF was added (12.5 vol) to dilute the mixture. The reaction mixture was washed with water (10.0 vol) 2 times. 2-MeTHF was added to bring the total volume of reaction to 40.0 vol (˜16.5 vol charged). To this solution was added NaOMe/MeOH (1.7 equiv) to perform the methanolysis. The reaction was stirred for no less than 1.0 hour, and checked for completion by HPLC. Once complete, the reaction was quenched with 1 N HCl (10.0 vol), and washed with 0.1 N HCl (10.0 vol). The organic solution was polish filtered to remove any particulates and placed in a second reactor. The filtered solution was concentrated at no more than 45° C. (jacket temperature) and no less than 8.0° C. (internal reaction temperature) under reduced pressure to 20 vol. CH₃CN was added to 40 vol and the solution concentrated at no more than 45° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was added followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 0.0° C.+/−5° C. over 5 hours, and filtered. The cake was washed with 0.0° C.+/−5.0° C. CH₃CN (5 vol) 4 times. The resulting solid (Compound 1) was dried in a vacuum oven at no more than 50.0° C. ¹H NMR (400 MHz, DMSO-d₆) δ 12.8 (s, 1H), 11.8 (s, 1H), 9.2 (s, 111), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

An alternative synthesis of Compound 1 is depicted in Scheme 1-7.

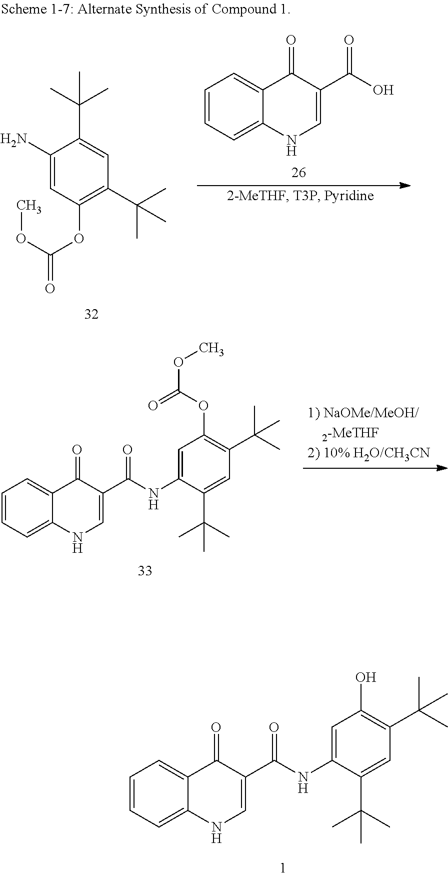

### Example 1g

### N-(2,4-di-tert-butyl-5-hydroxyphenyl)-4-oxo-1,4-dihydroquinoline-3-carboxamide (1)

4-Oxo-1,4-dihydroquinoline-3-carboxylic acid 26 (1.0 eq) and 5-amino-2,4-di-tert-butylphenyl methyl carbonate 32 (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 20° C.+/−5° C. 2-MeTHF was added (12.5 vol) to dilute the mixture. The reaction mixture was washed with water (10.0 vol) 2 times and 2-MeTHF (16.5 vol) was charged to the reactor. This solution was charged with 30% w/w NaOMe/MeOH (1.7 equiv) to perform the methanolysis. The reaction was stirred at 25.0° C.+/−5.0° C. for no less than 1.0 hour, and checked for completion by HPLC. Once complete, the reaction was quenched with 1.2 N HCl/H₂O (10.0 vol), and washed with 0.1 N HCl/H₂O (10.0 vol). The organic solution was polish filtered to remove any particulates and placed in a second reactor.

The filtered solution was concentrated at no more than 45° C. (jacket temperature) and no less than 8.0° C. (internal reaction temperature) under reduced pressure to 20 vol. CH₃CN was added to 40 vol and the solution concentrated at no more than 45° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was charged followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 20 to 25° C. over 5 hours, and filtered. The cake was washed with CH₃CN (5 vol) heated to 20 to 25° C. 4 times. The resulting solid (Compound 1) was dried in a vacuum oven at no more than 50.0° C. ¹H NMR (400 MHz, DMSO-d₆) δ 12.8 (s, 1H), 11.8 (s, 1H), 9.2 (s, 1H), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

### III. Solid Forms of Compound 1

### III.A. Compound 1 Form C

### III.A.1. Characterization and Embodiments of Compound 1 Form C

XRPD (X-Ray Powder Diffraction)

The XRPD patterns were acquired at room temperature in reflection mode using a Bruker D8 Advance diffractometer equipped with a sealed tube copper source and a Vantec-1 detector. The X-ray generator was operating at a voltage of 40 kV and a current of 40 mA. The data were recorded in a θ-θ scanning mode over the range of 3°-40° 2θ with a step size of 0.014° and the sample spinning at 15 rpm.

In one aspect, Compound 1 is in Form C. In one embodiment, of this aspect, the invention includes crystalline N-[2,4-bis(1,1-dimethylethyl)-5-hydroxyphenyl]-1,4-dihydro-4-oxoquinoline-3-carboxamide (Compound 1) characterized as Form C.

In one embodiment of this aspect, Form C is characterized by a peak having a 2-Theta value from about 6.0 to about 6.4 degrees in an XRPD pattern. In a further embodiment, Form C is characterized by a peak having a 2-Theta value from about 7.3 to about 7.7 degrees in an XRPD pattern. In a further embodiment, Form C is characterized by a peak having a 2-Theta value from about 8.1 to about 8.5 degrees in an XRPD pattern. In a further embodiment, Form C is characterized by a peak having a 2-Theta value from about 12.2 to about 12.6 degrees in an XRPD pattern. In a further embodiment, Form C is characterized by a peak having a 2-Theta value from about 14.4 to about 14.8 degrees in an XRPD pattern. In a further embodiment, Form C is characterized by a peak having a 2-Theta value from about 17.7 to about 18.1 degrees in an XRPD pattern. In a further embodiment, Form C is characterized by a peak having a 2-Theta value from about 20.3 to about 20.7 degrees in an XRPD pattern. In a further embodiment, Form C is characterized by a peak having a 2-Theta value from about 20.7 to about 21.1 degrees in an XRPD pattern.

In another embodiment, Form C is characterized by a peak having a 2-Theta value of about 6.2 degrees in an XRPD pattern. In a further embodiment, Form C is characterized by a peak having a 2-Theta value of about 7.5 degrees in an XRPD pattern. In a further embodiment, Form C is characterized by a peak having a 2-Theta value of about 8.3 degrees in an XRPD pattern. In a further embodiment, Form C is characterized by a peak having a 2-Theta value of about 12.4 degrees in an XRPD pattern. In a further embodiment, Form C is characterized by a peak having a 2-Theta value of about 14.6 degrees in an XRPD pattern. In a further embodiment, Form C is characterized by a peak having a 2-Theta value of about 17.9 degrees in an XRPD pattern. In a further embodiment, Form C is characterized by a peak having a 2-Theta value of about 20.5 degrees in an XRPD pattern. In a further embodiment, Form C is characterized by a peak having a 2-Theta value of about 20.9 degrees in an XRPD pattern.

In another embodiment, Form C is characterized by one or more peaks in an XRPD pattern selected from about 6.2, about 7.5, about 8.3, about 12.4, about 14.6, about 17.9, about 20.5 and about 20.9 degrees as measured on a 2-Theta scale.

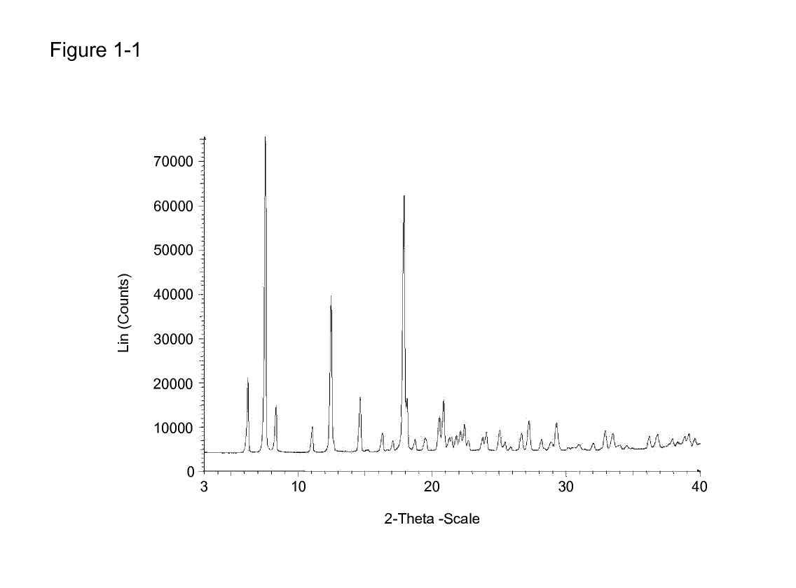

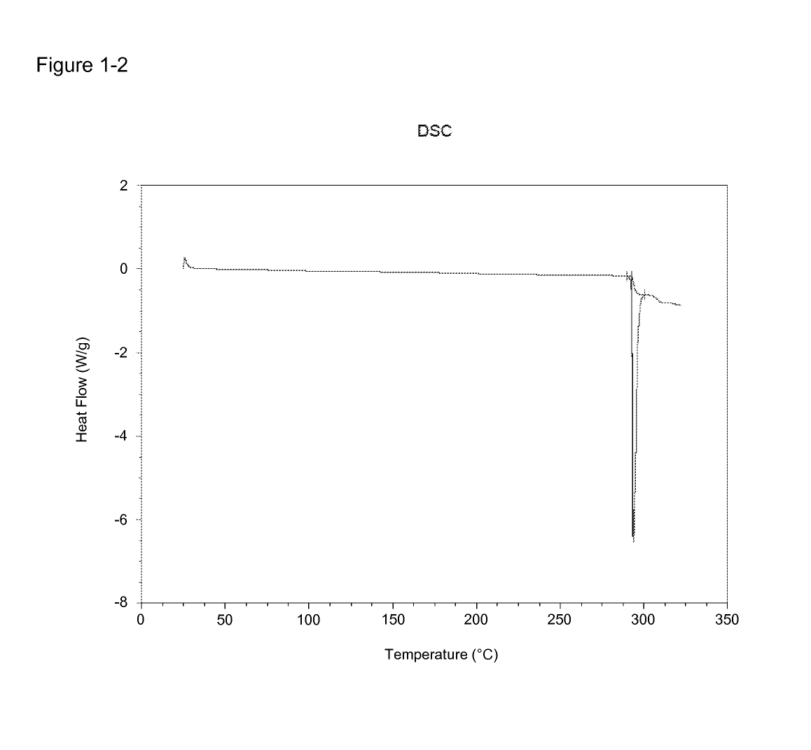

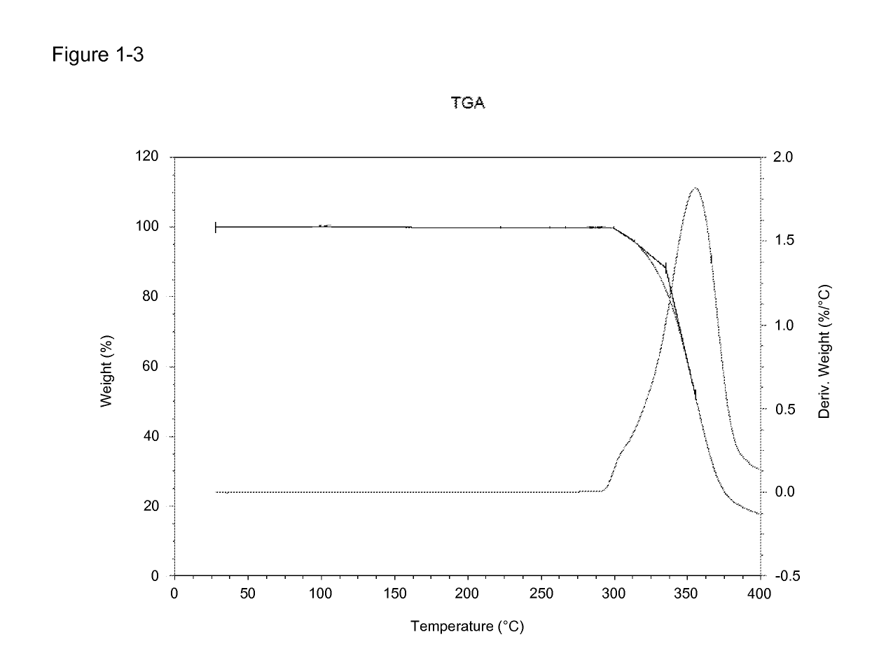

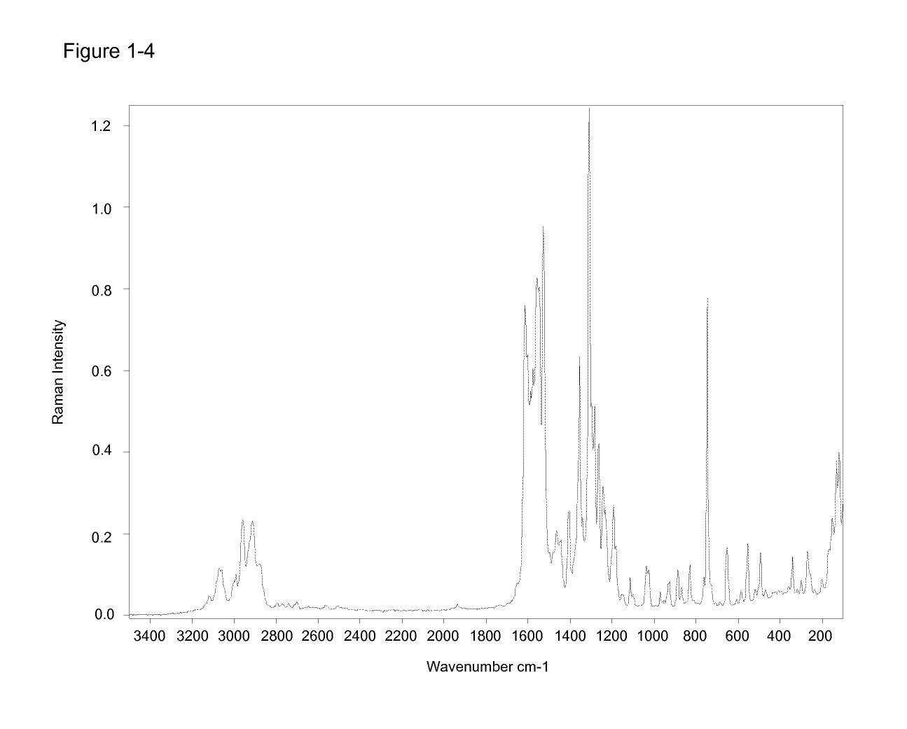

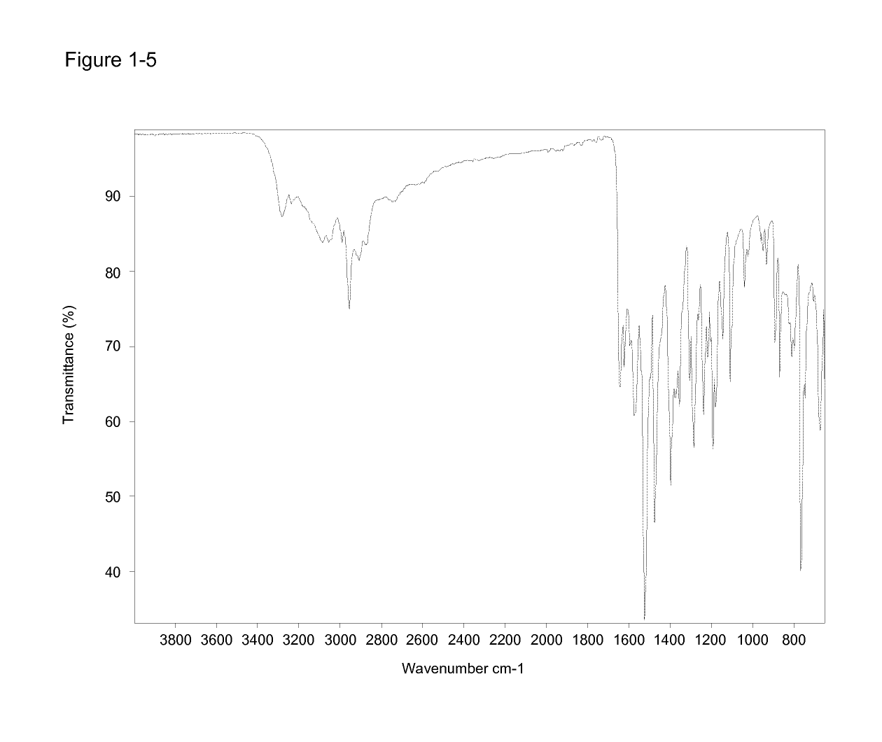

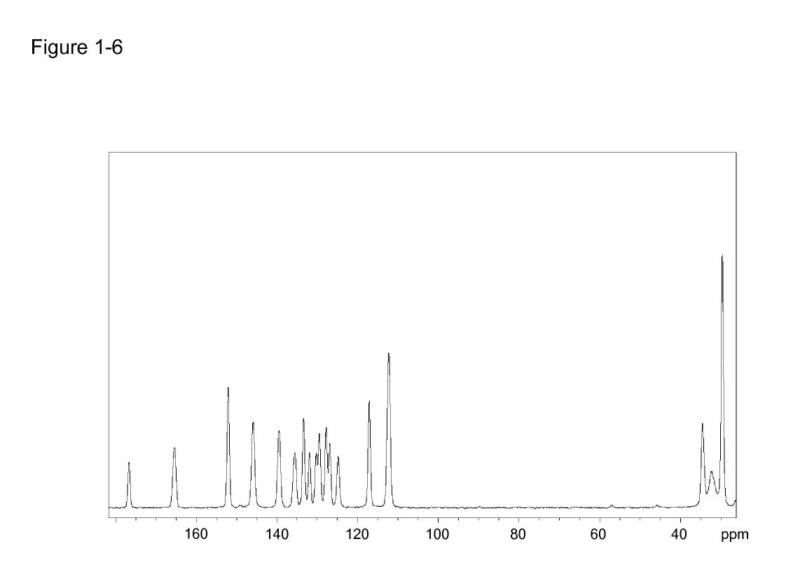

In still another embodiment, Form C is characterized by all of the following peaks in an XRPD pattern: about 6.2, about 7.5, about 8.3, about 12.4, about 14.6, about 17.9, about 20.5 and about 20.9 degrees as measured on a 2-Theta scale. Compound 1 Form C can be characterized by the X-Ray powder diffraction pattern depicted in FIG. 1-1. Representative peaks as observed in the XRPD pattern are provided in Table 1-1a and Table 1-1b below. Each peak described in Table 1-1a also has a corresponding peak label (A-H), which are used to describe some embodiments of the invention.

TABLE 1-1a
Representative XRPD peaks for Compound 1 Form C.

| Peak # | Angle 2-θ (°) | Peak Label |
|---|---|---|
| 1 | 6.2 | A |
| 2 | 7.5 | B |
| 3 | 8.3 | C |
| 4 | 12.4 | D |
| 5 | 14.6 | E |
| 6 | 17.9 | F |
| 7 | 20.5 | G |
| 8 | 20.9 | H |

In another embodiment, Form C can be characterized by an X-Ray powder diffraction pattern having the representative peaks listed in Table 1-1b.

TABLE 1-1b
Further representative XRPD peaks forForm C.

| Peak # | Angle 2-θ (°) |
|---|---|
| 1 | 6.2 |
| 2 | 7.5 |
| 3 | 8.3 |
| 4 | 11.0 |
| 5 | 12.4 |
| 6 | 14.6 |
| 7 | 16.3 |
| 8 | 17.1 |
| 9 | 17.9 |
| 10 | 18.1 |
| 11 | 18.7 |
| 12 | 19.5 |
| 13 | 20.5 |
| 14 | 20.9 |
| 15 | 21.3 |
| 16 | 21.5 |
| 17 | 21.8 |
| 18 | 22.1 |
| 19 | 22.4 |
| 20 | 22.7 |

In one aspect, Compound 1 Form C can be characterized by an X-Ray powder diffraction pattern having one or more of peaks A, B, C, D, E, F, G and H as described in Table 1-1a.

In one embodiment of this aspect, Form C is characterized by peak A. In another embodiment, Form C is characterized by peak B. In another embodiment, Form C is characterized by peak B. In another embodiment, Form C is characterized by peak C. In another embodiment, Form C is characterized by peak D. In another embodiment, Form C is characterized by peak E. In another embodiment, Form C is characterized by peak F. In another embodiment, Form C is characterized by peak G. In another embodiment, Form C is characterized by peak H.

In another embodiment of this aspect, Form C is characterized by an X-Ray powder diffraction pattern having one of the following groups of peaks as described in Table 1-1a: A and B; A and C; A and D; A and E; A and F; A and G; A and H; B and C; B and D; B and E; B and F; B and G; B and H; C and D; C and E; C and F; C and G; C and H; D and E; D and F; D and G; D and H; E and F; E and G; E and H; F and G; F and H; and G and H.

In another embodiment of this aspect, Form C is characterized by an X-Ray powder diffraction pattern having one of the following groups of peaks as described in Table 1-1a: A, B and C; A, B and D; A, B and E; A, B and F; A, B and G; A, B and H; A, C and D; A, C and E; A, C and F; A, C and G; A, C and H; A, D and E; A, D and F; A, D and G; A, D and H; A, E and F; A, E and G; A, E and H; A, F and G; A, F and H; A, G and H; B, C and D; B, C and E; B, C and F; B, C and G; B, C and H; B, D and E; B, D and F; B, D and G; B, D and H; B, E and F; B, E and G; B, E and H; B, F and G; B, F and H; B, G and H; C, D and E; C, D F; C, D and G; C, D and H; C, E and F; C, E and G; C, E and H; C, F and G; C, F and H; C, G and H; D, E and F; D, E and G; D, E and H; D, F and G; D, F and H; D, G and H; E, F and G; E, F and H, E, G and H; and F, G and H.

In another embodiment of this aspect, Form C is characterized by an X-Ray powder diffraction pattern having one of the following groups of peaks as described in Table 1-1a: A, B, C and D; A, B, C and E, A, B, C and F; A, B, C and G; A, B, C and H; A, B, D and E; A, B, D and F; A, B, D and G; A, B, D and H; A, B, E and F; A, B, E and G; A, B, E and H; A, B, F and G; A, B, F and H; A, B, G and H; A, C, D and E; A, C, D and F; A, C, D and G; A, C, D and H; A, C, E and F; A, C, E and G; A, C, E and H; A, C, F and G; A, C, F and H; A, C, G and H; A, D, F and G; A, D, F and H; A, D, G and H; A, E, F and G; A, E, F and H; A, E, G and H; A, F, G and H; B, C, D and E; B, C, D and F; B, C, D and G; B, C, D and H; B, C, E and F; B, C, E and G; B, C, E and H; B, C, F and G; B, C, F and H; B, C, G and H; B, D, E and F; B, D, E and G; B, D, E and H; B, D, F and G; B, D, F and H; B, D, G and H; B, E, F and G; B, E, F and H; B, E, G and H; B, F, G and H; C, D, E and F; C, D, E and G; C, D, E and H; C, D, F and G; C, D, F and H; C, D, G and H; C, E, F and G; C, E, F and H; C, E, G and H; C, F, G and H; D, E, F and G; D, E, F and H; D, E, G and H; D, F, G and H; and E, F, G and H.

In another embodiment of this aspect, Form C is characterized by an X-Ray powder diffraction pattern having one of the following groups of peaks as described in Table 1-1a: A, B, C, D and E; A, B, C, D and F; A, B, C, D and G; A, B, C, D and H; A, B, C, E and F; A, B, C, E and G; A, B, C, E and H; A, B, C, F and G; A, B, C, F and H; A, B, C, G and H; A, B, C, E and F; A, B, C, E and G; A, B, C, E and H; A, B, C, F and G; A, B, C, F and H; A, B, C, G and H; A, B, D, E and F; A, B, D, E and G; A, B, D, E and H; A, B, D, F and G; A, B, D, F and H; A, B, D, G and H; A, B, E, F and G; A, B, E, F and H; A, B, E, G and H; A, B, F, G and H; A, C, D, E and F; A, C, D, E and G; A, C, D, E and H; A, C, D, F and G; A, C, D, F and H; A, C, D, G and H; A, C, E, F and G; A, C, E, F and H; A, C, E, G and H; A, C, F, G and H; A, D, E, F and G; A, D, E, F and H; A, D, E, G and H; A, D, F, G and H; A, E, F, G and H; B, C, D, E and F; B, C, D, E and G; B, C, D, E and H; B, C, D, F and G; B, C, D, F and H; B, C, D, G and H; B, C, E, F and G; B, C, E, F and H; B, C, E, G and H; B, C, F, G and H; B, D, E, F and G; B, D, E, F and H; B, D, E, G and H; B, D, F, G and H; B, E, F, G and H; C, D, E, F and G; C, D, E, F and H; C, D, E, G and H; C, D, F, G and H; C, E, F, G and H; and D, E, F, G and H.

In another embodiment of this aspect, Form C is characterized by an X-Ray powder diffraction pattern having one of the following groups of peaks as described in Table 1-1a: A, B, C, D, E and F; A, B, C, D, E and G; A, B, C, D, E and H; A, B, C, D, F and G; A, B, C, D, F and H; A, B, C, D, G and H; A, B, C, E, F and G; A, B, C, E, F and H; A, B, C, E, G and H; A, B, C, F, G and H; A, B, D, E, F and G; A, B, D, E, F and H; A, B, D, E, G and H; A, B, D, F, G and H; A, B, E, F, G and H; A, C, D, E, F and G; A, C, D, E, F and H; A, C, D, E, G and H; A, C, D, F, G and H; A, C, E, F, G and H; A, D, E, F, G and H; B, C, D, E, F and G; B, C, D, E, F and H; B, C, D, E, G and H; B, C, D, F, G and H; B, C, E, F, G and H; B, D, E, F, G and H; and C, D, E, F, G and H.

In another embodiment of this aspect, Form C is characterized by an X-Ray powder diffraction pattern having one of the following groups of peaks as described in Table 1-1a: A, B, C, D, E, F and G; A, B, C, D, E, F and H; A, B, C, D, E, G and H; A, B, C, D, F, G and H; A, B, C, E, F, G and H; A, B, D, E, F, G and H; A, C, D, E, F, G and H; and B, C, D, E, F, G and H.

In another embodiment of this aspect, Form C is characterized by an X-Ray powder diffraction pattern having all of the following peaks as described in Table 1-1a: A, B, C, D, E, F, G and H.

In another aspect, Compound 1 Form C can be characterized by an X-Ray powder diffraction pattern having one or more of peaks that range in value within ±0.2 degrees of one or more of the peaks A, B, C, D, E, F, G and H as described in Table 1-1a. In one embodiment of this aspect, Form C is characterized by a peak within ±0.2 degrees of A. In another embodiment, Form C is characterized by a peak within ±0.2 degrees of B. In another embodiment, Form C is characterized by a peak within ±0.2 degrees of B. In another embodiment, Form C is characterized by a peak within ±0.2 degrees of C. In another embodiment, Form C is characterized by a peak within ±0.2 degrees of D. In another embodiment, Form C is characterized by a peak within ±0.2 degrees of E. In another embodiment, Form C is characterized by a peak within ±0.2 degrees of F. In another embodiment, Form C is characterized by a peak within ±0.2 degrees of G. In another embodiment, Form C is characterized by a peak within ±0.2 degrees of H.

In another embodiment of this aspect, Form C is characterized by an X-Ray powder diffraction pattern having one of the following groups of peaks as described in Table 1-1a: A and B; A and C; A and D; A and E; A and F; A and G; A and H; B and C; B and D; B and E; B and F; B and G; B and H; C and D; C and E; C and F; C and G; C and H; D and E; D and F; D and G; D and H; E and F; E and G; E and H; F and G; F and H; and G and H, wherein each peak in the group is within ±0.2 degrees of the corresponding value described in Table 1-1a.

In another embodiment of this aspect, Form C is characterized by an X-Ray powder diffraction pattern having one of the following groups of peaks as described in Table 1-1a: A, B and C; A, B and D; A, B and E; A, B and F; A, B and G; A, B and H; A, C and D; A, C and E; A, C and F; A, C and G; A, C and H; A, D and E; A, D and F; A, D and G; A, D and H; A, E and F; A, E and G; A, E and H; A, F and G; A, F and H; A, G and H; B, C and D; B, C and E; B, C and F; B, C and G; B, C and H; B, D and E; B, D and F; B, D and G; B, D and H; B, E and F; B, E and G; B, E and H; B, F and G; B, F and H; B, G and H; C, D and E; C, D F; C, D and G; C, D and H; C, E and F; C, E and G; C, E and H; C, F and G; C, F and H; C, G and H; D, E and F; D, E and G; D, E and H; D, F and G; D, F and H; D, G and H; E, F and G; E, F and H, E, G and H; and F, G and H, wherein each peak in the group is within ±0.2 degrees of the corresponding value described in Table 1-1a.

In another embodiment of this aspect, Form C is characterized by an X-Ray powder diffraction pattern having one of the following groups of peaks as described in Table 1-1a: A, B, C and D; A, B, C and E, A, B, C and F; A, B, C and G; A, B, C and H; A, B, D and E; A, B, D and F; A, B, D and G; A, B, D and H; A, B, E and F; A, B, E and G; A, B, E and H; A, B, F and G; A, B, F and H; A, B, G and H; A, C, D and E; A, C, D and F; A, C, D and G; A, C, D and H; A, C, E and F; A, C, E and G; A, C, E and H; A, C, F and G; A, C, F and H; A, C, G and H; A, D, F and G; A, D, F and H; A, D, G and H; A, E, F and G; A, E, F and H; A, E, G and H; A, F, G and H; B, C, D and E; B, C, D and F; B, C, D and G; B, C, D and H; B, C, E and F; B, C, E and G; B, C, E and H; B, C, F and G; B, C, F and H; B, C, G and H; B, D, E and F; B, D, E and G; B, D, E and H; B, D, F and G; B, D, F and H; B, D, G and H; B, E, F and G; B, E, F and H; B, E, G and H; B, F, G and H; C, D, E and F; C, D, E and G; C, D, E and H; C, D, F and G; C, D, F and H; C, D, G and H; C, E, F and G; C, E, F and H; C, E, G and H; C, F, G and H; D, E, F and G; D, E, F and H; D, E, G and H; D, F, G and H; and E, F, G and H, wherein each peak in the group is within ±0.2 degrees of the corresponding value described in Table 1-1a.

In another embodiment of this aspect, Form C is characterized by an X-Ray powder diffraction pattern having one of the following groups of peaks as described in Table 1-1a: A, B, C, D and E; A, B, C, D and F; A, B, C, D and G; A, B, C, D and H; A, B, C, E and F; A, B, C, E and G; A, B, C, E and H; A, B, C, F and G; A, B, C, F and H; A, B, C, G and H; A, B, C, E and F; A, B, C, E and G; A, B, C, E and H; A, B, C, F and G; A, B, C, F and H; A, B, C, G and H; A, B, D, E and F; A, B, D, E and G; A, B, D, E and H; A, B, D, F and G; A, B, D, F and H; A, B, D, G and H; A, B, E, F and G; A, B, E, F and H; A, B, E, G and H; A, B, F, G and H; A, C, D, E and F; A, C, D, E and G; A, C, D, E and H; A, C, D, F and G; A, C, D, F and H; A, C, D, G and H; A, C, E, F and G; A, C, E, F and H; A, C, E, G and H; A, C, F, G and H; A, D, E, F and G; A, D, E, F and H; A, D, E, G and H; A, D, F, G and H; A, E, F, G and H; B, C, D, E and F; B, C, D, E and G; B, C, D, E and H; B, C, D, F and G; B, C, D, F and H; B, C, D, G and H; B, C, E, F and G; B, C, E, F and H; B, C, E, G and H; B, C, F, G and H; B, D, E, F and G; B, D, E, F and H; B, D, E, G and H; B, D, F, G and H; B, E, F, G and H; C, D, E, F and G; C, D, E, F and H; C, D, E, G and H; C, D, F, G and H; C, E, F, G and H; and D, E, F, G and H, wherein each peak in the group is within ±0.2 degrees of the corresponding value described in Table 1-1a.

In another embodiment of this aspect, Form C is characterized by an X-Ray powder diffraction pattern having one of the following groups of peaks as described in Table 1-1a: A, B, C, D, E and F; A, B, C, D, E and G; A, B, C, D, E and H; A, B, C, D, F and G; A, B, C, D, F and H; A, B, C, D, G and H; A, B, C, E, F and G; A, B, C, E, F and H; A, B, C, E, G and H; A, B, C, F, G and H; A, B, D, E, F and G; A, B, D, E, F and H; A, B, D, E, G and H; A, B, D, F, G and H; A, B, E, F, G and H; A, C, D, E, F and G; A, C, D, E, F and H; A, C, D, E, G and H; A, C, D, F, G and H; A, C, E, F, G and H; A, D, E, F, G and H; B, C, D, E, F and G; B, C, D, E, F and H; B, C, D, E, G and H; B, C, D, F, G and H; B, C, E, F, G and H; B, D, E, F, G and H; and C, D, E, F, G and H, wherein each peak in the group is within ±0.2 degrees of the corresponding value described in Table 1-1a.

In another embodiment of this aspect, Form C is characterized by an X-Ray powder diffraction pattern having one of the following groups of peaks as described in Table 1-1a: A, B, C, D, E, F and G; A, B, C, D, E, F and H; A, B, C, D, E, G and H; A, B, C, D, F, G and H; A, B, C, E, F, G and H; A, B, D, E, F, G and H; A, C, D, E, F, G and H; and B, C, D, E, F, G and H, wherein each peak in the group is within ±0.2 degrees of the corresponding value described in Table 1-1a.

In another embodiment of this aspect, Form C is characterized by an X-Ray powder diffraction pattern having all of the following peaks as described in Table 1-1a: A, B, C, D, E, F, G and H, wherein each peak in the group is within ±0.2 degrees of the corresponding value described in Table 1-1a.

Rietveld Refinement of Form C (Compound 1) from Powder

High resolution data were collected for a crystalline powder sample of Compound 1 Form C (Collection performed at the European Synchrotron Radiation Facility, Grenoble, France) at the beamline ID31. The X-rays are produced by three 11-mm-gap ex-vacuum undulators. The beam is monochromated by a cryogenically cooled double-crystal monochromator (Si 111 crystals). Water-cooled slits define the size of the beam incident on the monochromator, and of the monochromatic beam transmitted to the sample in the range of 0.5-2.5 mm (horizontal) by 0.1-1.5 mm (vertical). The wavelength used for the experiment was 1.29984(3) Å.

The powder diffraction data were processed and indexed using Materials Studio (Reflex module). The structure was solved using PowderSolve module of Materials Studio. The resulting solution was assessed for structural viability and subsequently refined using Rietveld refinement procedure.

The structure was solved and refined in a centrosymmetric space group P2₁/c using simulated annealing algorithm. The main building block in form C is a dimer composed of two Compound 1 molecules related to each other by a crystallographic inversion center and connected via a pair of hydrogen bonds between the hydroxyl and the amide carbonyl group. These dimers are then further arranged into infinite chains and columns through hydrogen bonding, π-π stacking and van der Waals interactions. Two adjacent columns are oriented perpendicular to each other, one along the crystallographic direction a, the other along b. The columns are connected with each other through van der Waals interactions.

The 4-oxo-1H-quinoline group is locked in a nearly coplanar conformation with the amide group via an intramolecular hydrogen bond. Owing to the centrosymmetric space group, Form C structure contains two Compound 1 molecular conformations related to one another by rotation around the C1-N12 bond.

A powder pattern calculated from the crystal structure of form C and an experimental powder pattern recorded on powder diffractometer using a flat sample in reflectance mode have been compared. The peak positions are in excellent agreement. Some discrepancies in intensities of some peaks exist and are due to preferred orientation of crystallites in the flat sample.

The results of refinement, instrument setup, radiation details, lattice parameters of the resulting crystal are listed below.

TABLE 1-2
Results of refinement:

| | | | |
|---|---|---|---|
| Final R_{wp}: | 10.24% | Final Rₚ: | 7.27% |
| Final R_{wp} (without | 15.98% | Final CMACS: | 0.09% |
| background): | | | |

TABLE 1-3
Results of further refinement:

| | | | |
|---|---|---|---|
| Final R_{wp}: | 10.50% | Final Rₚ: | 7.49% |
| Final R_{wp} (without | 16.41% | Final CMACS: | 0.09% |
| background): | | | |

TABLE 1-4
Setup

| | | | |
|---|---|---|---|
| 2θ Range | 1.00-50.00 | Step Size | 0.003 |
| (degrees): | | (degrees): | |
| Excluded Regions: | — | | |

TABLE 1-5
Radiation

| Type: | X-ray | Source: | Synchrotron |
|---|---|---|---|
| λ₁ (Å): | 1.299840 | Monochromator: | Double |
| Anom. | No | Angle: | 50.379 |
| Dispersion: | | Polarization: | 0.950 |

TABLE 1-6
Lattice Parameters (Lattice Type: Monoclinic;
Space Group: P2₁/c

| Parameter | Value | Refined? |
|---|---|---|
| a | 12.211 Å | Yes |
| b | 5.961 Å | Yes |
| c | 32.662 Å | Yes |
| α | 90.00° | No |
| β | 119.62° | Yes |
| γ | 90.00° | No |

In one embodiment, the crystal structure of Compound 1 Form C has a monoclinic lattice type. In another embodiment, the crystal structure of Compound 1 Form C has a P2₁/c space group. In another embodiment, the crystal structure of Compound 1 Form C has a monoclinic lattice type and a P2₁/c space group.

In one embodiment, the crystal structure of Compound 1 Form C has the following unit cell dimensions:
a=12.211 Angstroms
b=5.961 Angstroms
c=32.662 Angstroms
α=90.00°
β=119.62°
γ=90.00°

In one aspect, the invention includes Pharmaceutical compositions including Compound 1 Form C and a pharmaceutically acceptable adjuvant or carrier. In one embodiment, Compound 1 Form C can be formulated in a pharmaceutical composition, in some instances, with another therapeutic agent, for example another therapeutic agent for treating cystic fibrosis or a symptom thereof.

Processes for preparing Compound 1 Form C are exemplified herein.

Methods of treating a CFTR mediated disease, such as cystic fibrosis, in a patient include administering to said patient Compound 1 Form C or a pharmaceutical composition comprising Compound 1 Form C.

Compound 1 Form C can be also characterized by an endotherm beginning at 292.78° C., that plateaus slightly and then peaks at 293.83° C. as measured by DSC (FIG. 1-2). Further, this endotherm precedes an 85% weight loss, as measured by TGA (FIG. 1-3), which is attributed to chemical degradation.

Compound 1 Form C can be characterized by a FT-IR spectrum as depicted in FIG. 1-5 and by Raman spectroscopy as depicted by FIG. 1-4.

Compound 1 Form C can be characterized by solid state NMR spectrum as depicted in FIG. 1-6.

Processes for preparing Compound 1 Form C are exemplified below.

### III.A.2. Synthesis of Compound 1 Form C

Compound 1 Form C was prepared by adding an excess of optionally recrystallized Compound 1, prepared as provided in Section II.A.3, into acetonitrile, stirring at 90° C. for 3 days, and cooling to room temperature. The product was harvested by filtration, and the purity of the Compound was confirmed using SSNMR. The recrystallization procedure is reproduced below for convenience.

Recrystallization of Compound 1

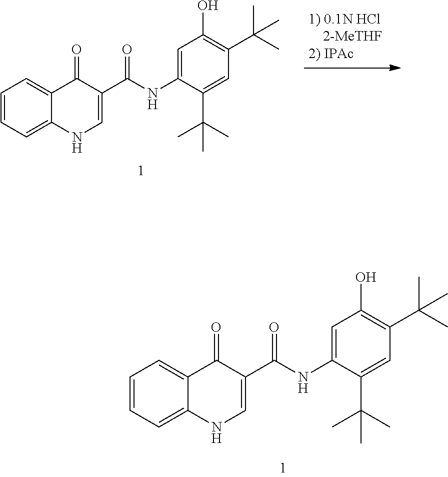

Compound 1 (1.0 eq) was charged to a reactor. 2-MeTHF (20.0 vol) was added followed by 0.1N HCl (5.0 vol). The biphasic solution was stirred and separated and the top organic phase was washed twice more with 0.1N HCl (5.0 vol). The organic solution was polish filtered to remove any particulates and placed in a second reactor. The filtered solution was concentrated at no more than 35° C. (jacket temperature) and no more than 8.0° C. (internal reaction temperature) under reduced pressure to 10 vol. Isopropyl acetate (IPAc) (10 vol) was added and the solution concentrated at no more than 35° C. (jacket temperature) and no more than 8.0° C. (internal reaction temperature) to 10 vol. The addition of IPAc and concentration was repeated 2 more times for a total of 3 additions of IPAc and 4 concentrations to 10 vol. After the final concentration, 10 vol of IPAc was charged and the slurry was heated to reflux and maintained at this temperature for 5 hours. The slurry was cooled to 0.0° C.+/−5° C. over 5 hours and filtered. The cake was washed with IPAc (5 vol) once. The resulting solid was dried in a vacuum oven at 50.0° C.+/−5.0° C.

Methods & Materials

Differential Scanning Calorimetry (DSC)

The DSC traces of Form C were obtained using TA Instruments DSC Q2000 equipped with Universal Analysis 2000 software. An amount (3-8 mg) of Compound 1 Form C was weighed into an aluminum pan and sealed with a pinhole lid. The sample was heated from 25° C. to 325° C. at 10° C./min. The sample exhibited high melting points which is consistent with highly crystalline material. In one embodiment, the melting range is about 293.3 to about 294.7° C. In a further embodiment, the melting range is about 293.8° C. to about 294.2° C. In another embodiment, the onset temperature range is about 292.2° C. to about 293.5° C. In a further embodiment, the onset temperature range is about 292.7° C. to about 293.0° C.

Thermogravimetric Analysis (TGA)

TGA was conducted on a TA Instruments model Q5000. An amount (3-5 mg) of Compound 1 Form C was placed in a platinum sample pan and heated at 10° C./min from room temperature to 400° C. Data were collected by Thermal Advantage Q Series™ software and analyzed by Universal Analysis 2000 software.

XRPD (X-Ray Powder Diffraction)

As stated previously, the XRPD patterns were acquired at room temperature in reflection mode using a Bruker D8 Advance diffractometer equipped with a sealed tube copper source and a Vantec-1 detector. The X-ray generator was operating at a voltage of 40 kV and a current of 40 mA. The data were recorded in a θ-θ scanning mode over the range of 3°-40° 2θ with a step size of 0.014° and the sample spinning at 15 rpm.

Raman and FTIR Spectroscopy

Raman spectra for Compound 1, Form C was acquired at room temperature using the VERTEX 70 FT-IR spectrometer coupled to a RAMII FT-Raman module. The sample was introduced into a clear vial, placed in the sample compartment and analyzed using the parameters outlined in the table below.

Raman Parameters

| Parameter | Setting | |
|---|---|---|
| Beam splitter | CaF₂ | |
| Laser frequency | 9395.0 | cm⁻¹ |
| Laser power | 1000 | mW |
| Save data from | 3501 to 2.94 | cm⁻¹ |
| Resolution | 4 | cm⁻¹ |
| Sample scan time | 64 | scans |

The FTIR spectra for Compound 1, Form C was acquired at room temperature using the Bruker VERTEX 70 FT-IR spectrometer using the parameters described in the table below.

FTIR Parameters

| Parameter | Setting | |
|---|---|---|
| Scan range | 4000-650 | cm⁻¹ |
| Resolution | 4 | cm⁻¹ |

| | |
|---|---|
| Scans sample | 16 |
| Scans background | 16 |
| Sampling mode | ATR, single reflection ZnSe |

TABLE 1-7
FTIR and Raman peak assignments for Compound 1, Form C:

| | FTIR | Raman |
|---|---|---|
| | Wavenumber | Wavenumber |
| Peak assignments | Intensity | Intensity |
| N—H str in | 3281 m | Not observed |
| —C(═O)-NHR trans | | |
| Unsaturated C—H | 3085 m, 3056 m | 3071 w, 2991 w |
| str-substituted | | |
| aromatic and olefin | | |
| Aliphatic C—H str | 2991 m, 2955 m, | 2959 w, 2913 w, |
| | 2907 m, 2876 m | 2878 w |
| Amide C═O str + | 1643 s | Not observed |
| Conjugated ketone C═O str | | |
| Olefin C═C conjugated | Not observed | 1615 s |
| with C═O | | |
| Amide II in | 1524 vs | 1528 s |
| —C(═O)-NHR trans | | |
| Benzene ring str | 1475 s | Not observed |
| Amide III in | 1285 s | 1310 vs |
| —C(═O)-NHR trans | | |
| Aromatic C—H wag | 765 vs | Not observed |
| Aromatic in-plane | Not observed | 748 s |
| bend modes | | |

vs = very strong
s = strong,
m = medium,
w = weak intensity.

SSNMR (Solid State Nuclear Magnetic Resonance Spectroscopy)

Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed of 12.0 kHz. The proton relaxation time was first measured using ¹H MAS T₁ saturation recovery relaxation experiment in order to set up proper recycle delay of the ^{—}C cross-polarization (CP) MAS experiment. The CP contact time of carbon CPMAS experiment was set to 2 ms. A CP proton pulse with linear ramp (from 50% to 100%) was employed. The Hartmann-Hahn match was optimized on external reference sample (glycine). TPPM15 decoupling sequence was used with the field strength of approximately 100 kHz. Some peaks from a ¹³C SSNMR spectrum of Compound 1 Form C are given in Table 1-1c.

TABLE 1-1c
Listing of some of the SSNMR peaks for Form C.
Compound 1 Form C

| | Chemical | | |
|---|---|---|---|
| Peak # | Shift [ppm] | Intensity | Peak Label |
| 1 | 176.5 | 17.95 | A |
| 2 | 165.3 | 23.73 | B |
| 3 | 152.0 | 47.53 | C |
| 4 | 145.8 | 33.97 | D |
| 5 | 139.3 | 30.47 | E |
| 6 | 135.4 | 21.76 | F |
| 7 | 133.3 | 35.38 | G |
| 8 | 131.8 | 21.72 | H |
| 9 | 130.2 | 21.45 | I |
| 10 | 129.4 | 29.31 | J |
| 11 | 127.7 | 31.54 | K |
| 12 | 126.8 | 25.44 | L |
| 13 | 124.8 | 20.47 | M |
| 14 | 117.0 | 42.4 | N |
| 15 | 112.2 | 61.08 | O |
| 16 | 34.5 | 33.34 | P |
| 17 | 32.3 | 14.42 | Q |
| 18 | 29.6 | 100 | R |

In some embodiments, the ¹³C SSNMR spectrum of Compound 1 Form C is includes one or more of the following peaks: 176.5 ppm, 165.3 ppm, 152.0 ppm, 145.8 ppm, 139.3 ppm, 135.4 ppm, 133.3 ppm, 131.8 ppm, 130.2 ppm, 129.4 ppm, 127.7 ppm, 126.8 ppm, 124.8 ppm, 117.0 ppm, 112.2 ppm, 34.5 ppm, 32.3 ppm and 29.6 ppm.

In some embodiments, the ¹³C SSNMR spectrum of Compound 1 Form C includes all of the following peaks: 152.0 ppm, 135.4 ppm, 131.8 ppm, 130.2 ppm, 124.8 ppm, 117.0 ppm and 34.5 ppm.

In some embodiments, the ¹³C SSNMR spectrum of Compound 1 Form C includes all of the following peaks: 152.0 ppm, 135.4 ppm, 131.8 ppm and 117.0 ppm.

In some embodiments, the 13C SSNMR spectrum of Compound 1 Form C includes all of the following peaks: 135.4 ppm and 131.8 ppm.

In some embodiments, the SSNMR of Compound 1 Form C includes a peak at about 152.0 ppm, about 135.4, about 131.8 ppm, and about 117 ppm.

In one aspect, the invention includes Compound 1 Form C which is characterized by a ¹³C SSNMR spectrum having one or more of the following peaks: C, F, H, I, M, N and P, as described by Table 1-1c.

In one embodiment of this aspect, Form C is characterized by one peak in a ¹³C SSNMR spectrum, wherein the peak is selected from C, F, H, I, M, N and P, as described by Table 1-1c.

In another embodiment of this aspect, Form C is characterized by a ¹³C SSNMR spectrum having a group of peaks selected from C and F; C and H; C and N; F and H; F and N; and H and N, as described by Table 1-1c. In a further embodiment, the ¹³C SSNMR spectrum includes the peaks I, M and P as described by Table 1-1c.

In another embodiment of this aspect, Form C is characterized by a ¹³C SSNMR spectrum having a group of peaks selected from C, F and H; C, H and N; and F, H and N, as described by Table 1-1c. In a further embodiment, the ¹³C SSNMR spectrum includes the peaks I, M and P as described by Table 1-1c.

In another embodiment of this aspect, Form C is characterized by a ¹³C SSNMR spectrum having the following group of peaks: C, F, H and N, as described by Table 1-1c. In a further embodiment, the ¹³C SSNMR spectrum includes the peaks I, M and P as described by Table 1-1c.

In another embodiment of this aspect, Form C is characterized by a ¹³C SSNMR spectrum having a group of peaks selected from C and F; C and H, C and N; C and I; C and M; or C and P, as described by Table 1-1c. In another embodiment of this aspect, Form C is characterized by a ¹³C SSNMR spectrum having a group of peaks selected from F and H; F and N; F and I; F and M; or F and P as described by Table 1-1c. In another embodiment of this aspect, Form C is characterized by a ¹³C SSNMR spectrum having a group of peaks selected from H and N; H and I; H and M; or H and P as described by Table 1-1c. In another embodiment of this aspect, Form C is characterized by a ¹³C SSNMR spectrum having a group of peaks selected from N and I; N and M; or N and P as described by Table 1-1c. In another embodiment of this aspect, Form C is characterized by a ¹³C SSNMR spectrum having a group of peaks selected from I and M; I and P or M and P as described by Table 1-1c.

In another embodiment of this aspect, Form C is characterized by a ¹³C SSNMR spectrum having a group of peaks selected from C, F and H; C, F and N; C, F and I; C, F and M; or C, F and P as described by Table 1-1c. In another embodiment of this aspect, Form C is characterized by a ¹³C SSNMR spectrum having a group of peaks selected from C, H and N; C, H and I; C, H and M; or C, H and P as described by Table 1-1c. In another embodiment of this aspect, Form C is characterized by a ¹³C SSNMR spectrum having a group of peaks selected from C, N and I; C, N and M; or C, N and P as described by Table 1-1c. In another embodiment of this aspect, Form C is characterized by a ¹³C SSNMR spectrum having a group of peaks selected from C, I and M; or C, I and P as described by Table 1-1c. In another embodiment of this aspect, Form C is characterized by a ¹³C SSNMR spectrum having a group of peaks selected from C, M and P as described by Table 1-1c. In another embodiment of this aspect, Form C is characterized by a ¹³C SSNMR spectrum having a group of peaks selected from F, H, and N; F, H and I; F, H and M; or F, H and P as described by Table 1-1c. In another embodiment of this aspect, Form C is characterized by a ¹³C SSNMR spectrum having a group of peaks selected from F, N and I; F, N and M; or F, N and P as described by Table 1-1c. In another embodiment of this aspect, Form C is characterized by a ¹³C SSNMR spectrum having a group of peaks selected from F, I and M; or F, I and P as described by Table 1-1c. In another embodiment of this aspect, Form C is characterized by a ¹³C SSNMR spectrum having a group of peaks selected from F, M and P as described by Table 1-1c. In another embodiment of this aspect, Form C is characterized by a ¹³C SSNMR spectrum having a group of peaks selected from H, N and I; H, N and M; or H, N and P as described by Table 1-1c. In another embodiment of this aspect, Form C is characterized by a ¹³C SSNMR spectrum having a group of peaks selected from H, I and M; or H, I and P as described by Table 1-1c. In another embodiment of this aspect, Form C is characterized by a ¹³C SSNMR spectrum having a group of peaks selected from H, M and P as described by Table 1-1c. In another embodiment of this aspect, Form C is characterized by a ¹³C SSNMR spectrum having a group of peaks selected from N, I and M; or N, I and P as described by Table 1-1c. In another embodiment of this aspect, Form C is characterized by a ¹³C SSNMR spectrum having a group of peaks selected from N, M and P as described by Table 1-1c. In another embodiment of this aspect, Form C is characterized by a ¹³C SSNMR spectrum having a group of peaks selected from I, M and P as described by Table 1-1c.

In another embodiment of this aspect, Form C is characterized by a ¹³C SSNMR spectrum having a group of peaks selected from C, F, H, and N; C, F H, and I; C, F H, and M; or C, F H, and P as described by Table 1-1c. In another embodiment of this aspect, Form C is characterized by a ¹³C SSNMR spectrum having a group of peaks selected from F, H, N and I; F, H, N and M; or F, H, N and P as described by Table 1-1c. In another embodiment of this aspect, Form C is characterized by a ¹³C SSNMR spectrum having a group of peaks selected from H, N, I and M; H, N, I and P; or H, N, I and C as described by Table 1-1c. In another embodiment of this aspect, Form C is characterized by a ¹³C SSNMR spectrum having a group of peaks selected from N, I, M and P; N, I, M and C; or N, I, M and F as described by Table 1-1c. In another embodiment of this aspect, Form C is characterized by a ¹³C SSNMR spectrum having a group of peaks selected from I, M, P and C; I, M, P and F; I, M, P and H as described by Table 1-1c.

In another embodiment of this aspect, Form C is characterized by a ¹³C SSNMR spectrum having a group of peaks selected from C, H, N and I; C, H, N, and M; or C, H, N, and P as described by Table 1-1c. In another embodiment of this aspect, Form C is characterized by a ¹³C SSNMR spectrum having a group of peaks selected from C, N, I and M; C, N, I and P; or C, N, I and F as described by Table 1-1c. In another embodiment of this aspect, Form C is characterized by a ¹³C SSNMR spectrum having a group of peaks selected from C, I, M and P; C, I, M and F; or C, I, M and H as described by Table 1-1c. In another embodiment of this aspect, Form C is characterized by a ¹³C SSNMR spectrum having a group of peaks selected from C, M, P and F; C, M, P and H; or C, M, P and N as described by Table 1-1c. In another embodiment of this aspect, Form C is characterized by a ¹³C SSNMR spectrum having a group of peaks selected from F, N, I and M; F, N, I and P; or F, N, I and C as described by Table 1-1c. In another embodiment of this aspect, Form C is characterized by a ¹³C SSNMR spectrum having a group of peaks selected from F, I, M and P; F, I, M and C; F, I, M and H; or F, I, M and N as described by Table 1-1c. In another embodiment of this aspect, Form C is characterized by a ¹³C SSNMR spectrum having a group of peaks selected from F, M, P and C; F, M, P and H; or F, M, P and N as described by Table 1-1c. In another embodiment of this aspect, Form C is characterized by a ¹³C SSNMR spectrum having a group of peaks selected from H, I, M and P; H, I, M and C; or H, I, M and F as described by Table 1-1c. In another embodiment of this aspect, Form C is characterized by a ¹³C SSNMR spectrum having a group of peaks selected from N, M, P and C; N, M, P and F; or N, M, P and H as described by Table 1-1c. In another embodiment of this aspect, Form C is characterized by a ¹³C SSNMR spectrum having a group of peaks selected from N, M, C and F; or N, M, C and H as described by Table 1-1c. In another embodiment of this aspect, Form C is characterized by a ¹³C SSNMR spectrum having a group of peaks selected from N, M, F and P as described by Table 1-1c. In another embodiment of this aspect, Form C is characterized by a ¹³C SSNMR spectrum having a group of peaks selected from N, M, H and P as described by Table 1-1c. In another embodiment of this aspect, Form C is characterized by a ¹³C SSNMR spectrum having a group of peaks selected from C, H, I and P; C, F, I and P; C, F, N and P or F, H, I and P as described by Table 1-1c.

In another embodiment of this aspect, Form C is characterized by a ¹³C SSNMR spectrum having a group of peaks selected from C, F, H, N and I; C, F, H, N and M; or C, F, H, N and P; C, F, H, I and M; C, F, H, I and P; C, F, H, M and P; C, F, N, I and M; C, F, N, I and P; C, F, N, M and P; C, H, N, I and M; C, H, N, I and P; C, H, N, M and P; C, H, I, M and P; F, H, N, I and M; F, H, N, I and P; F, H, N, M and P; F, H, I, M and P; F, N, I, M and P or H, N, I, M and P as described by Table 1-1c.

In another embodiment of this aspect, Form C is characterized by a ¹³C SSNMR spectrum having a group of peaks selected from C, F, H, N and I; C, F, H, N and M; or C, F, H, N and P as described by Table 1-1c. In another embodiment of this aspect, Form C is characterized by a ¹³C SSNMR spectrum having a group of peaks selected from C, H, N, I and M; or C, H, N, I and P as described by Table 1-1c. In another embodiment of this aspect, Form C is characterized by a ¹³C SSNMR spectrum having a group of peaks selected from C, N, I, M and P; or C, N, I, M and F as described by Table 1-1c. In another embodiment of this aspect, Form C is characterized by a ¹³C SSNMR spectrum having a group of peaks selected from C, I, M, P and F; or C, I, M, P and H as described by Table 1-1c. In another embodiment of this aspect, Form C is characterized by a ¹³C SSNMR spectrum having a group of peaks selected from C, M, P, F and H; or C, M, P, F and N as described by Table 1-1c. In another embodiment of this aspect, Form C is characterized by a ¹³C SSNMR spectrum having a group of peaks selected from C, P, F, H and I; or C, P, F, H and M as described by Table 1-1c. In another embodiment of this aspect, Form C is characterized by a ¹³C SSNMR spectrum having a group of peaks selected from F, H, N, I and M; or F, H, N, I and P as described by Table 1-1c. In another embodiment of this aspect, Form C is characterized by a ¹³C SSNMR spectrum having a group of peaks selected from F, N, I, M and P; or F, N, I, M and C as described by Table 1-1c. In another embodiment of this aspect, Form C is characterized by a ¹³C SSNMR spectrum having a group of peaks selected from F, I, M, C and H; F, I, M, C and N as described by Table 1-1c. In another embodiment of this aspect, Form C is characterized by a “C SSNMR spectrum having a group of peaks selected from F, M, P, C and H; F, M, P, C and N , N, I and M; or F, H, N, I and P as described by Table 1-1c. In another embodiment of this aspect, Form C is characterized by a ¹³C SSNMR spectrum having a group of peaks selected from H, N, I M, and P as described by Table 1-1c. In another embodiment of this aspect, Form C is characterized by a ¹³C SSNMR spectrum having a group of peaks selected from H, I M, P and F as described by Table 1-1c. In another embodiment of this aspect, Form C is characterized by a ¹³C SSNMR spectrum having a group of peaks selected from H, M, P, C and F as described by Table 1-1c. In another embodiment of this aspect, Form C is characterized by a ¹³C SSNMR spectrum having a group of peaks selected from H, P, C, F and I as described by Table 1-1c.

In another embodiment of this aspect, Form C is characterized by a ¹³C SSNMR spectrum having a group of peaks selected from C, F, H, N, I, and M; or C, F, H, N, I and P as described by Table 1-1c. In another embodiment of this aspect, Form C is characterized by a ¹³C SSNMR spectrum having a group of peaks selected from F, H, N, I, M and P as described by Table 1-1c. In another embodiment of this aspect, Form C is characterized by a ¹³C SSNMR spectrum having a group of peaks selected from H, N, I, M, P and C as described by Table 1-1c. In another embodiment of this aspect, Form C is characterized by a ¹³C SSNMR spectrum having a group of peaks selected from N, I, M, P, C and F as described by Table 1-1c. In another embodiment of this aspect, Form C is characterized by a ¹³C SSNMR spectrum having a group of peaks selected from M, P, C, F, H and N as described by Table 1-1c.

In another embodiment of this aspect, Form C is characterized by a ¹³C SSNMR spectrum having a group of peaks selected from C, F, H, N, I, and M; C, F, H, N, I and P; C, F, H, N, M and P; C, F, H, I, M and P; C, F, N, I, M and P; C, H, N, I, M and P or F, H, N, I, M and P as described by Table 1-1c.

In another embodiment of this aspect, Form C is characterized by a 13C SSNMR spectrum having a group of peaks selected from C, F, H, N, I, M and P as described by Table 1-1c.

### IV. Formulations of Compound 1

In some embodiments, Compound 1 is formulated as provided herein, and may include any solid forms of Compound 1.

### IV.A. Compound 1 First Formulation

### IV.A.1. Embodiments of Compound 1 First Formulation

In one embodiment, the Compound 1 Formulation comprises:
(i) Compound 1;
(ii) PEG 400; and
(iii) PVP K30.

In another embodiment, the Compound 1 Formulation comprises:
(i) Compound 1 or a pharmaceutically acceptable salt thereof;
(ii) A liquid PEG (polyethylene glycol polymer) that has an average molecular weight of between about 200 and about 600; and
(iii) Optionally, PVP.

In another embodiment, the Compound 1 Formulation comprises:
(i) Compound 1 or a pharmaceutically acceptable salt thereof;
(ii) a suitable liquid PEG; and
(iii) optionally, a suitable viscosity enhancing agent.

As used herein, the phrase “suitable liquid PEG” means a polyethylene glycol polymer that is in liquid form at ambient temperature and is amenable for use in a pharmaceutical composition. Such suitable polyethylene glycols are well known in the art; see, e.g., http://www.medicinescomplete.com/mc/excipients/current, which is incorporated herein by reference. Exemplary PEGs include low molecular weight PEGs such as PEG 200, PEG 300, PEG 400, etc. The number that follows the term “PEG” indicates the average molecular weight of that particular polymer. E.g., PEG 400 is a polyethylene glycol polymer wherein the average molecular weight of the polymer therein is about 400.

In one embodiment, said suitable liquid PEG has an average molecular weight of from about 200 to about 600. In another embodiment, said suitable liquid PEG is PEG 400 (for example a PEG having a molecular weight of from about 380 to about 420 g/mol).

In another embodiment, the present invention provides a pharmaceutical composition comprising Compound 1 or a pharmaceutically acceptable salt thereof; propylene glycol; and, optionally, a suitable viscosity enhancing agent.

In another embodiment, the pharmaceutical formulations of the present invention comprise a suitable viscosity enhancing agent. In one embodiment, the suitable viscosity enhancing agent is a polymer soluble in PEG. Such suitable viscosity enhancing agents are well known in the art, e.g., polyvinyl pyrrolidine (hereinafter “PVP”). PVP is characterized by its viscosity in aqueous solution, relative to that of water, expressed as a K-value (denoted as a suffix, e.g., PVP K20), in the range of from about 10 to about 120. See, e.g., http://www.medicinescomplete.com/mc/excipients/current. Embodiments of PVP useful in the present invention have a K-value of about 90 or less. An exemplary such embodiment is PVP K30.

In one embodiment, the Compound 1 formulation comprises:
(i) Compound 1 or a pharmaceutically acceptable salt thereof;
(ii) PEG 400; and
(iii) PVP K30.

In another embodiment, Compound 1 is present in an amount from about 0.01% w/w to about 6.5% w/w.

In another embodiment, the present invention provides a pharmaceutical formulation, wherein said PEG is present in an amount from about 87.5% w/w to about 99.99% w/w.

In another embodiment, the PVP K30 is present in an amount between 0% w/w to about 6% w/w.

In another embodiment, the formulation comprises PEG 400 (e.g., from about 97.8 to about 98.0% w/w, for example, about 97.88% w/w), PVP K30 (e.g., from about 1.9 to about 2.1% w/w, for example, about 2.0% w/w), and Compound 1 (e.g., from about 0.10 to about 0.15% w/w, for example, about 0.13% w/w).

In another embodiment, the formulation comprises PEG 400 (e.g., from about 97.5 to about 98.0% w/w, for example, about 97.75% w/w), PVP K30 (e.g., from about 1.8 to about 2.2% w/w, for example, about 2.0% w/w), and Compound 1 (e.g., from about 0.2 to about 0.3% w/w, for example, about 0.25% w/w).

In another embodiment, the formulation comprises PEG 400 (e.g., from about 97.2 to about 97.8, for example, about 97.50% w/w), PVP K30 (e.g., from about 1.8 to about 2.2% w/w, for example, about 2.0% w/w), and Compound 1 (e.g., from about 0.4 to about 0.6% w/w, for example, about 0.50% w/w).

In another embodiment, the formulation comprises PEG 400 (e.g., from about 96.5 to about 97.5% w/w, for example, about 97.0% w/w), PVP K30 (e.g., from about 1.8 to about 2.2% w/w, for example, about 2.0% w/w), and Compound 1 (e.g., from about 0.9 to about 1.1% w/w, for example, about 1.0% w/w).

In another embodiment, formulation comprises PEG 400 (e.g., from about 96.60 to about 96.65% w/w, for example, about 96.63% w/w), PVP K30 (e.g., from about 1.8 to about 2.2% w/w, for example, about 2.0% w/w), and Compound 1 (e.g., from about 1.30 to about 1.45% w/w, for example, about 1.38% w/w).

In another embodiment, the formulation comprises PEG 400 (e.g., from about 96.0 to about 96.3% w/w, for example, about 96.12% w/w), PVP K30 (e.g., from about 1.8 to about 2.0% w/w, for example, about 2.0% w/w), and Compound 1 (e.g., from about 1.8 to about 2.2% w/w, for example, about 1.88% w/w).

In another embodiment, the formulation comprises PEG 400 (e.g., from about 95.5 to about 96.0% w/w, for example, about 95.75% w/w), PVP K30 (e.g., from about 1.8 to about 2.2% w/w, for example, about 2.0% w/w), and Compound 1 (e.g., from about 2.0 to about 2.5% w/w, for example, about 2.25% w/w).

In another embodiment, the formulation comprises PEG 400 (e.g., from about 95 to about 96% w/w, for example, about 95.5% w/w), PVP K30 (e.g., from about 1.8 to about 2.2% w/w, for example, about 2.0% w/w), and Compound 1 (e.g., from about 2.3 to about 2.7% w/w, for example, about 2.50 w/w).

In another embodiment, the formulation comprises PEG 400 (e.g., from about 94.5 to about 94.8, for example, about 94.63% w/w), PVP K30 (e.g., from about 1.8 to about 2.2% w/w, for example, about 2.0% w/w), and Compound 1 (e.g., from about 3.5 to about 4.0% w/w, for example, about 3.38% w/w).

In another embodiment, the formulation comprises PEG 400 (e.g., from about 93.5 to about 94.5% w/w, for example, about 94.0% w/w), PVP K30 (e.g., from about 1.8 to about 2.2% w/w, for example, about 2.0% w/w), and Compound 1 (e.g., from about 3.7 to about 4.3% w/w, for example, about 4.0% w/w).

In one embodiment, the formulation comprises:
(i) Compound 1 or a pharmaceutically acceptable salt thereof;
(ii) a suitable PEG lipid; and
(iii) PVP.

In some embodiments, the PEG lipid has an average molecular weight of from about 400 to about 600, for example, PEG 400. In some embodiments, the PVP is PVP K30.

The formulation comprises a therapeutically effective amount of Compound 1. The phrase “therapeutically effective amount” is that amount effective for treating or lessening the severity of any of the diseases, conditions, or disorders recited below.

### IV.A.2. Preparation of Compound 1 First Formulation

### Materials:

A Glass bottle for formulation preparation (250 cc amber glass with Teflon lined lid)
Glass bottle for dose confirmation sample (30 cc amber glass with Teflon lined lid)
Stir Plate with temperature probe (ensure probe has been cleaned)
New magnetic stir bar
Spatulas for dispensing excipient and active.

### Step 1:

To a clean 250 cc amber glass bottle add the stir bar to the bottle and record the tare weight of the bottle, stir bar, label and cap. Tare the bottle with the label and stir bar.

### Step 2:

Dispense targeted amount of PEG400 into the bottle and accurately weigh. Place the bottle on stir plate and stir to form a small vortex at the surface of the liquid (˜300-500 rpm or as necessary). Insert the cleaned temperature probe into the liquid to a depth of ˜1 cm and raise the setpoint of the heater to 40° C. Cover the bottle opening with aluminum foil. Allow the PEG400 to stabilize at 40+/−5° C.

### Step 3:

Dispense the required amount of PVP K30 and add to the stirring PEG400. Add the PVP in a slow stream (over ˜2-3 minutes) and allow the particles to disperse. If the particles clump, the dissolution will take longer. Cover the bottle opening with foil and continue stirring the mixture at 40+/−5° C. The mixture should be sampled at 10 minutes using a small transfer pipette to determine if the PVP has completely dissolved. The stirring solution should also be examined for large, undissolved clumps. If the solution is clear, proceed to the next step. If undissolved polymer remains, continue stirring. Check for dissolution every 10 minutes, with a maximum stirring time of 30 minutes total. When complete dissolution is observed, proceed to the next step. If complete dissolution is not observed within 30 minutes after PVP addition, terminate preparation, discard the material, and start the preparation from the beginning.

### Step 4:

Dispense the required amount of Compound 1 and add to the stirred PEG/PVP solution in a slow stream. Cover the bottle opening with foil and continue stirring the mixture at 40+/−5° C. The mixture should be sampled after 30 minutes using a small transfer pipette to determine if the Compound 1 has completely dissolved. If the solution is clear after 30 minutes, proceed to the next step. If undissolved Compound 1 remains, continue stirring. Check for dissolution every 30 minutes with a maximum stirring time of 300 minutes (5 hours) after addition of Compound 1. If complete dissolution is not observed within 300 minutes (5 hours) after addition of Compound 1, terminate preparation, discard the material, and start the preparation from the beginning.

Upon complete dissolution of the Compound 1, remove from the stir plate, and cap the bottle. The formulation should be maintained at room temperature until dosing, but must be dosed within 24 hours of preparation. If precipitation of Compound 1 is observed, do not dose the solution.

Using the above method, the following ten pharmaceutical formulations in Table 1-A were prepared.

TABLE 1-A

| Composi- | % PEG 400 | % PVP K30 | % Cmpd 1 | Amount of Cmpd 1 |
|---|---|---|---|---|
| tion # | w/w | w/w | w/w | per 20 g dose (mg) |
| 1 | 97.875 | 2.0 | 0.125 | 25 |
| 2 | 97.750 | 2.0 | 0.250 | 50 |
| 3 | 97.500 | 2.0 | 0.500 | 100 |
| 4 | 97.000 | 2.0 | 1.000 | 200 |
| 5 | 96.625 | 2.0 | 1.375 | 275 |
| 6 | 96.125 | 2.0 | 1.875 | 375 |
| 7 | 95.750 | 2.0 | 2.25 | 450 |
| 8 | 95.500 | 2.0 | 2.500 | 500 |
| 9 | 94.625 | 2.0 | 3.375 | 675 |
| 10 | 94.000 | 2.0 | 4.000 | 800 |

### IV.B. Compound 1 Tablet and SDD Formulation

### IV.B.1. Embodiments of Compound 1 Tablet and SDD Formulation

In one embodiment, the present invention provides a pharmaceutical composition comprising:
a. a solid dispersion of substantially amorphous Compound 1 and HPMCAS;
b. a filler;
c. a disintegrant;
d. a surfactant;
e. a binder;
f. a glidant; and
g. a lubricant,

wherein the solid dispersion comprises about 100 mg of substantially amorphous Compound 1.

In one embodiment, the present invention provides a pharmaceutical composition comprising:
a. a solid dispersion of substantially amorphous Compound 1 and HPMCAS;
b. a filler;
c. a disintegrant;
d. a surfactant;
e. a binder;
f. a glidant; and
g. a lubricant,

wherein the solid dispersion comprises about 150 mg of substantially amorphous Compound 1.

In one embodiment, the present invention provides a pharmaceutical composition comprising:
a. a solid dispersion of amorphous Compound 1 and HPMCAS;
b. a filler;
c. a disintegrant;
d. a surfactant;
e. a binder;
f. a glidant; and
g. a lubricant,

wherein the solid dispersion comprises about 100 mg of amorphous Compound 1.

In one embodiment, the present invention provides a pharmaceutical composition comprising:
a. a solid dispersion of amorphous Compound 1 and HPMCAS;
b. a filler;
c. a disintegrant;
d. a surfactant;
e. a binder;
f. a glidant; and
g. a lubricant,

wherein the solid dispersion comprises about 150 mg of amorphous Compound 1.

In some embodiments, the pharmaceutical composition comprises a solid dispersion a filler, a disintegrant, a surfactant, a binder, a glidant, and a lubricant, wherein the solid dispersion comprises from about 75 wt % to about 95 wt % (e.g., about 80 wt %) of Compound 1 by weight of the dispersion and a polymer.

In one embodiment, the pharmaceutical composition of the present invention comprises a solid dispersion of Compound 1. For example, the solid dispersion comprises substantially amorphous Compound 1, where Compound 1 is less than about 15% (e.g., less than about 10% or less than about 5%) crystalline, and at least one polymer. In another example, the solid dispersion comprises amorphous Compound 1, i.e., Compound 1 has about 0% crystallinity. The concentration of Compound 1 in the solid dispersion depends on several factors such as the amount of pharmaceutical composition needed to provide a desired amount of Compound 1 and the desired dissolution profile of the pharmaceutical composition.

In another embodiment, the pharmaceutical composition comprises a solid dispersion that contains substantially amorphous Compound 1 and HPMCAS, in which the solid dispersion has a mean particle diameter, measured by light scattering (e.g., using a Malvern Mastersizer available from Malvern Instruments in England) of greater than about 5 μm (e.g., greater than about 6 μm, greater than about 7 μm, greater than about 8 μm, or greater than about 10 μm). For example, the pharmaceutical composition comprises a solid dispersion that contains amorphous Compound 1 and HPMCAS, in which the solid dispersion has a mean particle diameter, measured by light scattering, of greater than about 5 μm (e.g., greater than about 6 μm, greater than about 7 μm, greater than about 8 μm, or greater than about 10 μm). In another example, the pharmaceutical composition comprises a solid dispersion comprising substantially amorphous Compound 1 and HPMCAS, in which the solid dispersion has a mean particle diameter, measured by light scattering, of from about 7 μm to about 25 μm. For instance, the pharmaceutical composition comprises a solid dispersion comprising amorphous Compound 1 and HPMCAS, in which the solid dispersion has a mean particle diameter, measured by light scattering, of from about 7 μm to about 25 μm. In yet another example, the pharmaceutical composition comprises a solid dispersion comprising substantially amorphous Compound 1 and HPMCAS, in which the solid dispersion has a mean particle diameter, measured by light scattering, of from about 10 μm to about 35 μm. For instance, the pharmaceutical composition comprises a solid dispersion comprising amorphous Compound 1 and HPMCAS, in which the solid dispersion has a mean particle diameter, measured by light scattering, of from about 10 μm to about 35 μm. In another example, the pharmaceutical composition comprises a solid dispersion comprising substantially amorphous Compound 1 and HPMCAS, in which the solid dispersion has a bulk density of about 0.10 g/cc or greater (e.g., 0.15 g/cc or greater, 0.17 g/cc or greater). For instance, the pharmaceutical composition comprising a solid dispersion comprising amorphous Compound 1 and HPMCAS, in which the solid dispersion has a bulk density of about 0.10 g/cc or greater (e.g., 0.15 g/cc or greater, 0.17 g/cc or greater). In another instance, the pharmaceutical composition comprises a solid dispersion that comprises substantially amorphous Compound 1 and HPMCAS, in which the solid dispersion has a bulk density of from about 0.10 g/cc to about 0.45 g/cc (e.g., from about 0.15 g/cc to about 0.42 g/cc, or from about 0.17 g/cc to about 0.40 g/cc). In still another instance, the pharmaceutical composition comprises a solid dispersion that includes amorphous Compound 1 and HPMCAS, in which the solid dispersion has a bulk density of from about 0.10 g/cc to about 0.45 g/cc (e.g., from about 0.15 g/cc to about 0.42 g/cc, or from about 0.17 g/cc to about 0.40 g/cc). In another example, the pharmaceutical composition comprises a solid dispersion that comprises substantially amorphous Compound 1 and HPMCAS, in which the solid dispersion has a bulk density of from about 0.10 g/cc to about 0.45 g/cc (e.g., from about 0.15 g/cc to about 0.42 g/cc, or from about 0.17 g/cc to about 0.40 g/cc). For instance, the pharmaceutical composition includes a solid dispersion that comprises amorphous Compound 1 and HPMCAS, in which the solid dispersion has a bulk density of from about 0.10 g/cc to about 0.45 g/cc (e.g., from about 0.15 g/cc to about 0.42 g/cc, or from about 0.17 g/cc to about 0.40 g/cc).

Other solid dispersions comprise from about 65 wt % to about 95 wt % (e.g., from about 67 wt % to about 92 wt %, from about 70 wt % to about 90 wt %, or from about 72 wt % to about 88 wt %) of substantially amorphous Compound 1 by weight of the solid dispersion and from about 45 wt % to about 5 wt % of polymer (e.g., HPMCAS). For instance, the solid dispersion comprises from about 65 wt % to about 95 wt % (e.g., from about 67 wt % to about 92 wt %, from about 70 wt % to about 90 wt %, or from about 72 wt % to about 88 wt %) of amorphous Compound 1 by weight of the solid dispersion and from about 45 wt % to about 5 wt % of polymer (e.g., HPMCAS).

Suitable surfactants include sodium lauryl sulfate (SLS), sodium stearyl fumarate (SSF), polyoxyethylene 20 sorbitan mono-oleate (e.g., Tween™), any combination thereof, or the like. In one example, the solid dispersion comprises less than 5 wt % (less than 3.0 wt %, less than 1.5 wt %, or less than 1.0 wt %) of surfactant by weight of solid dispersion. In another example, the solid dispersion comprises from about 0.30 wt % to about 0.80 wt % (e.g., from about 0.35 wt % to about 0.70 wt %, from about 0.40 wt % to about 0.60 wt %, or from about 0.45 wt % to about 0.55 wt %) of surfactant by weight of solid dispersion.

In alternative embodiments, the solid dispersion comprises from about 45 wt % to about 85 wt % of substantially amorphous or amorphous Compound 1, from about 0.45 wt % to about 0.55 wt % of SLS, and from about 14.45 wt % to about 55.55 wt % of HPMCAS by weight of the solid dispersion. One exemplary solid dispersion contains about 80 wt % of substantially amorphous or amorphous Compound 1, about 19.5 wt % of HPMCAS, and about 0.5 wt % of SLS.

Fillers suitable for the present invention are compatible with the ingredients of the pharmaceutical composition, i.e., they do not substantially reduce the solubility, the hardness, the chemical stability, the physical stability, or the biological activity of the pharmaceutical composition. Exemplary fillers include lactose, sorbitol, celluloses, calcium phosphates, starches, sugars (e.g., mannitol, sucrose, or the like), or any combination thereof. In one embodiment, the pharmaceutical composition comprises at least one filler in an amount of at least about 10 wt % (e.g., at least about 20 wt %, at least about 25 wt %, or at least about 27 wt %) by weight of the composition. For example, the pharmaceutical composition comprises from about 10 wt % to about 60 wt % (e.g., from about 20 wt % to about 55 wt %, from about 25 wt % to about 50 wt %, or from about 27 wt % to about 45 wt %) of filler, by weight of the composition. In another example, the pharmaceutical composition comprises at least about 20 wt % (e.g., at least 25 wt % or at least 27 wt %) of lactose, by weight of the composition. In yet another example, the pharmaceutical composition comprises from about 20 wt % to about 60 wt % (e.g., from about 25 wt % to about 55 wt % or from about 27 wt % to about 45 wt %) of lactose, by weight of the composition.

Disintegrants suitable for the present invention enhance the dispersal of the pharmaceutical composition and are compatible with the ingredients of the pharmaceutical composition, i.e., they do not substantially reduce the chemical stability, the physical stability, the hardness, or the biological activity of the pharmaceutical composition. Exemplary disintegrants include sodium croscarmellose, sodium starch glycolate, or a combination thereof. In one embodiment, the pharmaceutical composition comprises disintegrant in an amount of about 10 wt % or less (e.g., about 7 wt % or less, about 6 wt % or less, or about 5 wt % or less) by weight of the composition. For example, the pharmaceutical composition comprises from about 1 wt % to about 10 wt % (e.g., from about 1.5 wt % to about 7.5 wt % or from about 2.5 wt % to about 6 wt %) of disintegrant, by weight of the composition. In another example, the pharmaceutical composition comprises about 10 wt % or less (e.g., 7 wt % or less, 6 wt % or less, or 5 wt % or less) of sodium croscarmellose, by weight of the composition. In yet another example, the pharmaceutical composition comprises from about 1 wt % to about 10 wt % (e.g., from about 1.5 wt % to about 7.5 wt % or from about 2.5 wt % to about 6 wt %) of sodium croscarmellose, by weight of the composition. In some examples, the pharmaceutical composition comprises from about 0.1% to about 10 wt % (e.g., from about 0.5 wt % to about 7.5 wt % or from about 1.5 wt % to about 6 wt %) of disintegrant, by weight of the composition. In still other examples, the pharmaceutical composition comprises from about 0.5% to about 10 wt % (e.g., from about 1.5 wt % to about 7.5 wt % or from about 2.5 wt % to about 6 wt %) of disintegrant, by weight of the composition.

Surfactants suitable for the present invention enhance the solubility of the pharmaceutical composition and are compatible with the ingredients of the pharmaceutical composition, i.e., they do not substantially reduce the chemical stability, the physical stability, the hardness, or the biological activity of the pharmaceutical composition. Exemplary surfactants include sodium lauryl sulfate (SLS), sodium stearyl fumarate (SSF), polyoxyethylene 20 sorbitan mono-oleate (e.g., Tween™), any combination thereof, or the like. In one embodiment, the pharmaceutical composition comprises a surfactant in an amount of about 10 wt % or less (e.g., about 5 wt % or less, about 2 wt % or less, about 1 wt % or less, about 0.8 wt % or less, or about 0.6 wt % or less) by weight of the composition. For example, the pharmaceutical composition includes from about 10 wt % to about 0.1 wt % (e.g., from about 5 wt % to about 0.2 wt % or from about 2 wt % to about 0.3 wt %) of surfactant, by weight of the composition. In another example, the pharmaceutical composition comprises 10 wt % or less (e.g., about 5 wt % or less, about 2 wt % or less, about 1 wt % or less, about 0.8 wt % or less, or about 0.6 wt % or less) of sodium lauryl sulfate, by weight of the composition. In yet another example, the pharmaceutical composition comprises from about 10 wt % to about 0.1 wt % (e.g., from about 5 wt % to about 0.2 wt % or from about 2 wt % to about 0.3 wt %) of sodium lauryl sulfate, by weight of the composition.

Binders suitable for the present invention enhance the tablet strength of the pharmaceutical composition and are compatible with the ingredients of the pharmaceutical composition, i.e., they do not substantially reduce the chemical stability, the physical stability, or the biological activity of the pharmaceutical composition. Exemplary binders include microcrystalline cellulose, dibasic calcium phosphate, sucrose, corn (maize) starch, modified cellulose (e.g., hydroxymethyl cellulose), or any combination thereof. In one embodiment, the pharmaceutical composition comprises a binder in an amount of at least about 1 wt % (e.g., at least about 10 wt %, at least about 15 wt %, at least about 20 wt %, or at least about 22 wt %) by weight of the composition. For example, the pharmaceutical composition comprises from about 5 wt % to about 50 wt % (e.g., from about 10 wt % to about 45 wt % or from about 20 wt % to about 45 wt %) of binder, by weight of the composition. In another example, the pharmaceutical composition comprises at least about 1 wt % (e.g., at least about 10 wt %, at least about 15 wt %, at least about 20 wt %, or at least about 22 wt %) of microcrystalline cellulose, by weight of the composition. In yet another example, the pharmaceutical composition comprises from about 5 wt % to about 50 wt % (e.g., from about 10 wt % to about 45 wt % or from about 20 wt % to about 45 wt %) of microcrystalline cellulose, by weight of the composition.

Glidants suitable for the present invention enhance the flow properties of the pharmaceutical composition and are compatible with the ingredients of the pharmaceutical composition, i.e., they do not substantially reduce the solubility, the hardness, the chemical stability, the physical stability, or the biological activity of the pharmaceutical composition. Exemplary glidants include colloidal silicon dioxide, talc, or a combination thereof. In one embodiment, the pharmaceutical composition comprises a glidant in an amount of 2 wt % or less (e.g., 1.75 wt %, 1.25 wt % or less, or 1.00 wt % or less) by weight of the composition. For example, the pharmaceutical composition comprises from about 2 wt % to about 0.05 wt % (e.g., from about 1.5 wt % to about 0.07 wt % or from about 1.0 wt % to about 0.09 wt %) of glidant, by weight of the composition. In another example, the pharmaceutical composition comprises 2 wt % or less (e.g., 1.75 wt %, 1.25 wt % or less, or 1.00 wt % or less) of colloidal silicon dioxide, by weight of the composition. In yet another example, the pharmaceutical composition comprises from about 2 wt % to about 0.05 wt % (e.g., from about 1.5 wt % to about 0.07 wt % or from about 1.0 wt % to about 0.09 wt %) of colloidal silicon dioxide, by weight of the composition.

Lubricants suitable for the present invention improve the compression and ejection of compressed pharmaceutical compositions from a die press and are compatible with the ingredients of the pharmaceutical composition, i.e., they do not substantially reduce the solubility, the hardness, or the biological activity of the pharmaceutical composition. Exemplary lubricants include magnesium stearate, stearic acid (stearin), hydrogenated oil, sodium stearyl fumarate, or any combination thereof. In one embodiment, the pharmaceutical composition comprises a lubricant in an amount of 2 wt % or less (e.g., 1.75 wt %, 1.25 wt % or less, or 1.00 wt % or less) by weight of the composition. For example, the pharmaceutical composition comprises from about 2 wt % to about 0.10 wt % (e.g., from about 1.5 wt % to about 0.15 wt % or from about 1.3 wt % to about 0.30 wt %) of lubricant, by weight of the composition. In another example, the pharmaceutical composition comprises 2 wt % or less (e.g., 1.75 wt %, 1.25 wt % or less, or 1.00 wt % or less) of magnesium stearate, by weight of the composition. In yet another example, the pharmaceutical composition comprises from about 2 wt % to about 0.10 wt % (e.g., from about 1.5 wt % to about 0.15 wt % or from about 1.3 wt % to about 0.30 wt %) of magnesium stearate, by weight of the composition.

Pharmaceutical compositions of the present invention can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability, the hardness, or the biological activity of the pharmaceutical composition. In one embodiment, the pharmaceutical composition comprises a colorant, a flavor, and/or a fragrance. For example, the pharmaceutical composition comprises less than about 1 wt % (e.g., less than about 0.75 wt % or less than about 0.5 wt %) of each optionally ingredient, i.e., colorant, flavor and/or fragrance, by weight of the composition. In another example, the pharmaceutical composition comprises less than about 1 wt % (e.g., less than about 0.75 wt % or less than about 0.5 wt %) of a colorant. In still another example, the pharmaceutical composition comprises less than about 1 wt % (e.g., less than about 0.75 wt % or less than about 0.5 wt %) of a blue colorant (e.g., FD&C Blue #1 and/or FD&C Blue #2 Aluminum Lake, commercially available from Colorcon, Inc. of West Point, Pa.)

In some embodiments, the pharmaceutical composition can be made into tablets and the tablets can be coated with a colorant and optionally labeled with a logo, other image and/or text using a suitable ink. In still other embodiments, the pharmaceutical composition can be made into tablets and the tablets can be coated with a colorant, waxed, and optionally labeled with a logo, other image and/or text using a suitable ink. Suitable colorants and inks are compatible with the ingredients of the pharmaceutical composition, i.e., they do not substantially reduce the solubility, the chemical stability, the physical stability, the hardness, or the biological activity of the pharmaceutical composition. The suitable colorants and inks can be any color and are water based or solvent based. In one embodiment, tablets made from the pharmaceutical composition are coated with a colorant and then labeled with a logo, other image, and/or text using a suitable ink. For example, tablets comprising pharmaceutical composition as described herein can be coated with about 3 wt % (e.g., less than about 6 wt % or less than about 4 wt %) of film coating comprising a colorant. The colored tablets can be labeled with a logo and text indicating the strength of the active ingredient in the tablet using a suitable ink. In another example, tablets comprising pharmaceutical composition as described herein can be coated with about 3 wt % (e.g., less than about 6 wt % or less than about 4 wt %) of a film coating comprising a blue colorant (e.g., OPADRY® II, commercially available from Colorcon, Inc. of West Point, Pa.). The colored tablets can be labeled with a logo and text indicating the strength of the active ingredient in the tablet using a black ink (e.g., Opacode® WB, commercially available from Colorcon, Inc. of West Point, Pa.). In another embodiment, tablets made from the pharmaceutical composition are coated with a colorant, waxed, and then labeled with a logo, other image, and/or text using a suitable ink. For example, tablets comprising pharmaceutical composition as described herein can be coated with about 3 wt % (e.g., less than about 6 wt % or less than about 4 wt %) of film coating comprising a colorant. The colored tablets can be waxed with Carnauba wax powder weighed out in the amount of about 0.01% w/w of the starting tablet core weight. The waxed tablets can be labeled with a logo and text indicating the strength of the active ingredient in the tablet using a suitable ink. In another example, tablets comprising pharmaceutical composition as described herein can be coated with about 3 wt % (e.g., less than about 6 wt % or less than about 4 wt %) of a film coating comprising a blue colorant (e.g., OPADRY® II, commercially available from Colorcon, Inc. of West Point, Pa.). The colored tablets can be waxed with Carnauba wax powder weighed out in the amount of about 0.01% w/w of the starting tablet core weight. The waxed tablets can be labeled with a logo and text indicating the strength of the active ingredient in the tablet using a black ink (e.g., Opacode® S-1-17823—a solvent based ink, commercially available from Colorcon, Inc. of West Point, Pa.).

Another exemplary pharmaceutical composition comprises from about 5 wt % to about 50 wt % (e.g., from about 5 wt % to about 25 wt %, from about 15 wt % to about 40 wt %, or from about 30 wt % to about 50 wt %) of a solid dispersion, by weight of the composition, comprising from about 70 wt % to about 90 wt % of substantially amorphous Compound 1, by weight of the dispersion, and from about 30 wt % to about 10 wt % of a polymer, by weight of the dispersion; from about 25 wt % to about 50 wt % of a filler; from about 1 wt % to about 10 wt % of a disintegrant; from about 2 wt % to about 0.3 wt % of a surfactant; from about 5 wt % to about 50 wt % of a binder; from about 2 wt % to about 0.05 wt % of a glidant; and from about 2 wt % to about 0.1 wt % of a lubricant. Or, the pharmaceutical composition comprises from about 5 wt % to about 50 wt % (e.g., from about 5 wt % to about 25 wt %, from about 15 wt % to about 40 wt %, or from about 30 wt % to about 50 wt %) of a solid dispersion, by weight of the composition, comprising from about 70 wt % to about 90 wt % of amorphous Compound 1, by weight of the dispersion, and from about 30 wt % to about 10 wt % of a polymer, by weight of the dispersion; from about 25 wt % to about 50 wt % of a filler; from about 1 wt % to about 10 wt % of a disintegrant; from about 2 wt % to about 0.3 wt % of a surfactant; from about 5 wt % to about 50 wt % of a binder; from about 2 wt % to about 0.05 wt % of a glidant; and from about 2 wt % to about 0.1 wt % of a lubricant.

In another pharmaceutical composition of the present invention, a caplet shaped pharmaceutical tablet composition having an initial hardness of between about 6 and 16 Kp comprises about 34.1 wt % of a solid dispersion by weight of the composition, wherein the dispersion comprises about 80 wt % of substantially amorphous Compound 1 by weight of the dispersion, about 19.5 wt % of HPMCAS by weight of the dispersion, and about 0.5 wt % SLS by weight of the dispersion; about 30.5 wt % of microcrystalline cellulose by weight of the composition; about 30.4 wt % of lactose by weight of the composition; about 3 wt % of sodium croscarmellose by weight of the composition; about 0.5 wt % of SLS by weight of the composition; about 0.5 wt % of colloidal silicon dioxide by weight of the composition; and about 1 wt % of magnesium stearate by weight of the composition. In some aspects, the caplet shaped pharmaceutical tablet composition contains 100 mg of Compound 1. In some further aspects, the caplet shaped pharmaceutical tablet composition comprises a colorant coated, a wax coating, and a printed logo or text. In some embodiments of this aspect, the caplet shaped pharmaceutical tablet includes a blue OPADRY® II coating and a water or solvent based ink logo or text. In some instances, the colorant coating is blue OPADRY® II. In some instances, the wax coating comprises Carnauba wax. In certain aspects, the ink for the printed logo or text is a solvent based ink. In some aspects, the caplet shaped pharmaceutical tablet composition contains 150 mg of Compound 1.

In still another pharmaceutical composition of the present invention, a pharmaceutical tablet composition having an initial hardness of between about 9 and 21 Kp comprises about 34.1 wt % of a solid dispersion by weight of the composition, wherein the dispersion comprises about 80 wt % of substantially amorphous Compound 1 by weight of the dispersion, about 19.5 wt % of HPMCAS by weight of the dispersion, and about 0.5 wt % SLS by weight of the dispersion; about 30.5 wt % of microcrystalline cellulose by weight of the composition; about 30.4 wt % of lactose by weight of the composition; about 3 wt % of sodium croscarmellose by weight of the composition; about 0.5 wt % of SLS by weight of the composition; about 0.5 wt % of colloidal silicon dioxide by weight of the composition; and about 1 wt % of magnesium stearate by weight of the composition. In some embodiments, the caplet shaped pharmaceutical tablet composition contains 150 mg of Compound 1. In some aspects, the caplet shaped pharmaceutical tablet composition further comprises a colorant coated, a wax coating, and a printed logo or text. In some instances, the tablet includes a blue OPADRY® II coating and a water or solvent based ink logo or text. In still other instances, the wax coating comprises Carnauba wax. In some embodiments, the ink for the printed logo or text is a solvent based ink. In some aspects, the caplet shaped pharmaceutical tablet composition contains 100 mg of Compound 1.

In another pharmaceutical composition of the present invention, a pharmaceutical composition comprises about 34.1 wt % of a solid dispersion by weight of the composition, wherein the dispersion comprises about 80 wt % of substantially amorphous Compound 1 by weight of the dispersion, about 19.5 wt % of HPMCAS by weight of the dispersion, and about 0.5 wt % SLS by weight of the dispersion; about 30.5 wt % of microcrystalline cellulose by weight of the composition; about 30.4 wt % of lactose by weight of the composition; about 3 wt % of sodium croscarmellose by weight of the composition; about 0.5 wt % of SLS by weight of the composition; about 0.5 wt % of colloidal silicon dioxide by weight of the composition; and about 1 wt % of magnesium stearate by weight of the composition. In some aspects, the pharmaceutical tablet contains 100 mg of Compound 1. In other embodiments, the pharmaceutical composition contains 150 mg of Compound 1. In some further aspects, the pharmaceutical composition is formed as a tablet and comprises a colorant coated, a wax coating, and a printed logo or text. In some embodiments of this aspect, the pharmaceutical tablet includes a blue OPADRY® II coating and a water or solvent based ink logo or text. In some instances, the colorant coating is blue OPADRY® II. In some instances, the wax coating comprises Carnauba wax. In certain aspects, the ink for the printed logo or text is a solvent based ink.

Another aspect of the present invention provides a pharmaceutical composition consisting of a tablet that includes a CF potentiator API (e.g., a solid dispersion of N-[2,4-bis(1,1-dimethylethyl)-5-hydroxyphenyl]-1,4-dihydro-4-oxoquinoline-3-carboxamide) and other excipients (e.g., a filler, a disintegrant, a surfactant, a binder, a glidant, a colorant, a lubricant, or any combination thereof), each of which is described above and in the Examples below, wherein the tablet has a dissolution of at least about 50% (e.g., at least about 60%, at least about 70%, at least about 80%, at least about 90%, or at least about 99%) in about 30 minutes. In one example, the pharmaceutical composition consists of a tablet that includes a CF potentiator API (e.g., a solid dispersion of Compound 1) and other excipients (e.g., a filler, a disintegrant, a surfactant, a binder, a glidant, a colorant, a lubricant, or any combination thereof), each of which is described above and in the Examples below, wherein the tablet has a dissolution of from about 50% to about 100% (e.g., from about 55% to about 95% or from about 60% to about 90%) in about 30 minutes. In another example, the pharmaceutical composition consists of a tablet that comprises a solid dispersion comprising substantially amorphous or amorphous Compound 1 and HPMCAS; and, a filler, a disintegrant, a surfactant, a binder, a glidant, and a lubricant, wherein the tablet has a dissolution of at least about 50% (e.g., at least about 60%, at least about 70%, at least about 80%, at least about 90%, or at least about 99%) in about 30 minutes. In still another example, the pharmaceutical composition consists of a tablet that comprises a solid dispersion comprising substantially amorphous or amorphous Compound 1 and HPMCAS; and, a filler, a disintegrant, a surfactant, a binder, a glidant, and a lubricant, wherein the tablet has a dissolution of from about 50% to about 100% (e.g., from about 55% to about 95% or from about 60% to about 90%) in about 30 minutes.

In one embodiment, the tablet comprises a solid dispersion comprising at least about 100 mg, or at least 150 mg of substantially amorphous or amorphous Compound 1; and HPMCAS and SLS.

Dissolution can be measured with a standard USP Type II apparatus that employs a dissolution media of 0.6% sodium lauryl sulfate dissolved in 900 mL of DI water, stirring at about 50-75 rpm at a temperature of about 37 ° C. A single experimental tablet is tested in each test vessel of the apparatus. Dissolution can also be measured with a standard USP Type II apparatus that employs a dissolution media of 0.7% sodium lauryl sulfate dissolved in 900 mL of 50 mM sodium phosphate buffer (pH 6.8), stirring at about 65 rpm at a temperature of about 37° C. A single experimental tablet is tested in each test vessel of the apparatus. Dissolution can also be measured with a standard USP Type II apparatus that employs a dissolution media of 0.5% sodium lauryl sulfate dissolved in 900 mL of 50 mM sodium phosphate buffer (pH 6.8), stirring at about 65 rpm at a temperature of about 37 ° C. A single experimental tablet is tested in each test vessel of the apparatus.

Another aspect of the present invention provides a pharmaceutical composition consisting of a tablet that comprises a CF potentiator API (e.g., a solid dispersion of Compound 1) and other excipients (e.g., a filler, a disintegrant, a surfactant, a binder, a glidant, a colorant, a lubricant, or any combination thereof), each of which is described above and in the Examples below, wherein the tablet has a hardness of at least about 5 Kp. In one example, the pharmaceutical composition consists of a tablet that comprises a CF potentiator API (e.g., a solid dispersion of Compound 1) and other excipients (e.g., a filler, a disintegrant, a surfactant, a binder, a glidant, a colorant, a lubricant, or any combination thereof), each of which is described above and in the Examples below, wherein the tablet has a hardness of at least about 5 Kp (e.g., at least about 5.5, at least about 6 Kp, or at least about 7 Kp).

### IV.B.2. Preparation of Compound 1 Tablet and SDD Formulation

Another aspect of the present invention provides a method of producing a pharmaceutical composition comprising providing an admixture of a solid dispersion of substantially amorphous or amorphous Compound 1, a binder, a glidant, a surfactant, a lubricant, a disintegrant, and a filler, and compressing the admixture into a tablet having a dissolution of at least about 50% in about 30 minutes.

Each of the ingredients of this admixture is described above and in the Examples below. Furthermore, the admixture can comprise optional additives such as one or more colorants, one or more flavors, and/or one or more fragrances as described above and in the Examples below. And, the relative concentrations (e.g., wt %) of each of these ingredients (and any optional additives) in the admixture is also presented above and in the Examples below. The ingredients constituting the admixture can be provided sequentially or in any combination of additions; and, the ingredients or combination of ingredients can be provided in any order. In one embodiment, the lubricant is the last component added to the admixture.

In one embodiment, the admixture comprises a solid dispersion of substantially amorphous Compound 1, a binder, a glidant, a surfactant, a lubricant, a disintegrant, and a filler, wherein each of these ingredients is provided in a powder form (e.g., provided as particles having a mean diameter, measured by light scattering, of 250 μm or less (e.g., 150 μm or less, 100 μm or less, 50 μm or less, 45 μm or less, 40 μm or less, or 35 μm or less)). For instance, the admixture comprises a solid dispersion of amorphous Compound 1, a binder, a glidant, a surfactant, a lubricant, a disintegrant, and a filler, wherein each of these ingredients is provided in a powder form (e.g., provided as particles having a mean diameter, measured by light scattering, of 250 μm or less (e.g., 150 μm or less, 100 μm or less, 50 μm or less, 45 μm or less, 40 μm or less, or 35 μm or less)).

In another embodiment, the admixture comprises a solid dispersion of substantially amorphous Compound 1, a binder, a glidant, a surfactant, a lubricant, a disintegrant, and a filler, wherein each of these ingredients is substantially free of water. Each of the ingredients comprises less than 5 wt % (e.g., less than 2 wt %, less than 1 wt %, less than 0.75 wt %, less than 0.5 wt %, or less than 0.25 wt %) of water by weight of the ingredient. For instance, the admixture comprises a solid dispersion of amorphous Compound 1, a binder, a glidant, a surfactant, a lubricant, a disintegrant, and a filler, wherein each of these ingredients is substantially free of water. Each of the ingredients comprises less than 5 wt % (e.g., less than 2 wt %, less than 1 wt %, less than 0.75 wt %, less than 0.5 wt %, or less than 0.25 wt %) of water by weight of the ingredient.

In another embodiment, compressing the admixture into a tablet is accomplished by filling a form (e.g., a mold) with the admixture and applying pressure to admixture. This can be accomplished using a die press or other similar apparatus. It is also noted that the application of pressure to the admixture in the form can be repeated using the same pressure during each compression or using different pressures during the compressions. In another example, the admixture is compressed using a die press that applies sufficient pressure to form a tablet having a dissolution of about 50% or more at about 30 minutes (e.g., about 55% or more at about 30 minutes or about 60% or more at about 30 minutes). For instance, the admixture is compressed using a die press to produce a tablet hardness of at least about 5 Kp (at least about 5.5 Kp, at least about 6 Kp, at least about 7 Kp, at least about 11 Kp, or at least 21Kp). In some instances, the admixture is compressed to produce a tablet hardness of between about 6 and 21 Kp.

In some embodiments, tablets comprising a pharmaceutical composition as described herein can be coated with about 3.0 wt % of a film coating comprising a colorant by weight of the tablet. In certain instances, the colorant suspension or solution used to coat the tablets comprises about 20% w/w of solids by weight of the colorant suspension or solution. In still further instances, the coated tablets can be labeled with a logo, other image or text.

In another embodiment, the method of producing a pharmaceutical composition comprises providing an admixture of a solid dispersion of substantially amorphous Compound 1, a binder, a glidant, a surfactant, a lubricant, a disintegrant, and a filler; mixing the admixture until the admixture is substantially homogenous, and compressing the admixture into a tablet as described above or in the Examples below. Or, the method of producing a pharmaceutical composition comprises providing an admixture of a solid dispersion of amorphous Compound 1, a binder, a glidant, a surfactant, a lubricant, a disintegrant, and a filler; mixing the admixture until the admixture is substantially homogenous, and compressing the admixture into a tablet as described above or in the Examples below. For example, the admixture is mixed by stirring, blending, shaking, or the like using hand mixing, a mixer, a blender, any combination thereof, or the like. When ingredients or combinations of ingredients are added sequentially, mixing can occur between successive additions, continuously throughout the ingredient addition, after the addition of all of the ingredients or combinations of ingredients, or any combination thereof. The admixture is mixed until it has a substantially homogenous composition.

Intermediate F

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Compound 1 were added according to the ratio 19.5 wt % hypromellose acetate succinate/0.5 wt % SLS/80 wt % Compound 1. The resulting mixture contained 10.5 wt % solids. The actual amounts of ingredients and solvents used to generate this mixture are recited in Table 1-F1.

TABLE 1-F1
Solid Spray Dispersion Ingredients for Intermediate F.

| | Units | Batch |
|---|---|---|
| Compound 1 | Kg | 70.0 |
| HPMCAS | Kg | 17.1 |
| SLS | Kg | 0.438 |
| Total Solids | Kg | 87.5 |
| MEK | Kg | 671 |
| Water | Kg | 74.6 |
| Total Solvents | Kg | 746 |
| Total Spray Solution Weight | Kg | 833 |

The mixture temperature was adjusted to a range of 20-45° C. and mixed until it was substantially homogenous and all components were substantially dissolved.

A spray drier, Niro PSD4 Commercial Spray Dryer, fitted with pressure nozzle (Spray Systems Maximum Passage series SK-MFP having orifice/core size 54/21) equipped with anti-bearding cap, was used under normal spray drying mode, following the dry spray process parameters recited in Table 1-F2.

TABLE 1-F2
Dry Spray Process Parameters Used to Generate Intermediate F.

| Parameter | Value | |
|---|---|---|
| Feed Pressure | 20 | bar |
| Feed Flow Rate | 92-100 | Kg/hr |
| Inlet Temperature | 93-99° | C. |
| Outlet Temperature | 53-57° | C. |
| Vacuum Dryer Temperature | 80° C. for 2 hours then | |
| | 110° C.(+/−5° C.) | |
| Vacuum Drying Time | 20-24 | hours |

A high efficiency cyclone separated the wet product from the spray gas and solvent vapors. The wet product contained 8.5-9.7% MEK and 0.56-0.83% Water and had a mean particle size of 17-19 um and a bulk density of 0.27-0.33 g/cc. The wet product was transferred to a 4000 L stainless steel double cone vacuum dryer for drying to reduce residual solvents to a level of less than about 5000 ppm and to generate dry Intermediate F. The dry Intermediate F contained <0.03% MEK and 0.3% Water.

Intermediate G

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Compound 1 were added according to the ratio 19.5 wt % hypromellose acetate succinate/0.5 wt % SLS/80 wt % Compound 1. The resulting mixture contained 10.5 wt % solids. The actual amounts of ingredients and solvents used to generate this mixture are recited in Table 1-G1.

TABLE 1-G1
Solid Spray Dispersion Ingredients for Intermediate G.

| | Units | Batch |
|---|---|---|
| Compound 1 | Kg | 24.0 |
| HPMCAS | Kg | 5.85 |
| SLS | Kg | 0.15 |
| Total Solids | Kg | 30.0 |
| MEK | Kg | 230.1 |
| Water | Kg | 25.6 |
| Total Solvents | Kg | 255.7 |
| Total Spray Solution Weight | Kg | 285.7 |

The mixture temperature was adjusted to a range of 20-45° C. and mixed until it was substantially homogenous and all components were substantially dissolved.

A spray drier, Niro Production Minor Spray Dryer, fitted with pressure nozzle (Spray Systems Maximum Passage series SK-MFP having orifice size 72) was used under normal spray drying mode, following the dry spray process parameters recited in Table 1-G2.

TABLE 1-G2
Dry Spray Process Parameters Used to Generate intermediate G.

| Parameter | Value | |
|---|---|---|
| Feed Pressure | 33 | bar |
| Feed Flow Rate | 18-24 | Kg/hr |
| Inlet Temperature | 82-84° | C. |
| Outlet Temperature | 44-46° | C. |
| Vacuum Dryer Temperature | 80° C. for 2 hours then | |
| | 110° C.(+/−5° C.) | |
| Vacuum Drying Time | 48 | hours |

A high efficiency cyclone separated the wet product from the spray gas and solvent vapors. The wet product contained 10.8% MEK and 0.7% Water and had a mean particle size of 19um and a bulk density of 0.32g/cc. The wet product was transferred to a 4000 L stainless steel double cone vacuum dryer for drying to reduce residual solvents to a level of less than about 5000 ppm and to generate dry Intermediate. The dry Intermediate G contained <0.05% MEK and 0.7% Water.

Intermediate H

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Compound 1 were added according to the ratio 19.5 wt % hypromellose acetate succinate/0.5 wt % SLS/80 wt % Compound 1. The actual amounts of ingredients and solvents used to generate this mixture are recited in Table 1-H1:

TABLE 1-H1
Solid Spray Dispersion Ingredients for Intermediate H.

| | Units | Batch |
|---|---|---|
| Compound 1 | Kg | 56.0 |
| HPMCAS | Kg | 13.65 |
| SLS | Kg | 0.35 |
| Total Solids | Kg | 70.0 |
| MEK | Kg | 509.73 |
| Water | Kg | 56.64 |
| Total Solvents | Kg | 566.40 |
| Total Spray Solution Weight | Kg | 636.40 |

The mixture temperature was adjusted to a range of 20-30° C. and mixed until it was substantially homogenous and all components were substantially dissolved.

A spray drier, Niro Production Minor Spray Dryer, fitted with pressure nozzle (Spray Systems Maximum Passage series SK-MFP having orifice size #52 or #54, e.g., about 1.39-1.62 mm) was used under normal spray drying mode, following the dry spray process parameters recited in Table 1-1-H2.

TABLE 1-H2
Dry Spray Process Parameters Used to Generate Intermediate H.

| Parameter | Value | |
|---|---|---|
| Feed Pressure | 20-50 | bar |
| Feed Flow Rate | 18-24 | Kg/hr |
| Inlet Temperature | −7 to 7° | C. |
| Outlet Temperature | 30-70° | C. |

A high efficiency cyclone separated the wet product from the spray gas and solvent vapors. The wet product contained approximately 10.8% MEK and 0.7% Water and had a mean particle size of about 19₁Am and a bulk density of about 0.33g/cc.

An inertial cyclone is used to separate the spray dried intermediate from the process gas and solvent vapors. Particle size is monitored on-line. The spray dried intermediate is collected in an intermediate bulk container. The process gas and solvent vapors are passed through a filter bag to collect the fine particles not separated by the cyclone. The resultant gas is condensed to remove process vapors and recycled back to the heater and spray dryer. The spray dried intermediate will be stored at less than 30° C., if secondary drying will occur in less than 24 hours or between 2-8° C., if secondary drying will occur in more than 24 hours.

Secondary drying occurs by charging a 4000-L biconical dryer having a jacket temperature between about 20-30° C. with the spray dried intermediate. The vacuum pressure, jacket temperature, and nitrogen bleed are set at between about −0.8 psig and about −1.0 psig, between about 80-120° C., and between about 0.5-8.0 m³/h, respectively. Agitation is set at 1 rpm. Bulk samples of the spray dried intermediate are tested for MEK (GC), every 4 hours until dry. The MEK drying rate is monitored on-line by GC-MS, calibrated for MEK concentration. Upon reaching a plateau in the drying of the residual MEK, heating in the biconical dryer is discontinued while continuing rotation until the spray dried intermediate reaches a temperature less than or equal to 50° C.

Although Intermediates F through H are described above as being formed, in part, by admixing the solid spray dispersion ingredients with application of heat to form a homogeneous mixture, the solid spray dispersion ingredients can also be mixed without application of heat to form a mixture of the solid spray dispersion ingredients.

### Tablets:

### Example 8

### Exemplary Tablet 9 (Formulated with HPMCAS Polymer to have 100 mg of Compound 1)

A batch of caplet-shaped tablets was formulated to have about 100 mg of Compound 1 per tablet using the amounts of ingredients recited in Table 1-8.

TABLE 1-8
Ingredients for Exemplary Tablet 9.

| | Percent Dose | Dose | Batch |
|---|---|---|---|
| Tablet Formulation | % Wt./Wt. | (mg) | (g) |
| Intermediate F | 34.09% | 125.1 | 23.86 |
| Microcrystalline cellulose | 30.51% | 112.0 | 21.36 |
| Lactose | 30.40% | 111.6 | 21.28 |
| Sodium croscarmellose | 3.000% | 11.01 | 2.100 |
| SLS | 0.500% | 1.835 | 0.3500 |
| Colloidal silicon dioxide | 0.500% | 1.835 | 0.3500 |
| Magnesium stearate | 1.000% | 3.670 | 0.7000 |
| Total | 100% | 367 | 70 |

The colloidal silicon dioxide (Cabot Cab-O-Sil® M-5P Fumed Silicon Dioxide) and the microcrystalline cellulose (FMC MCC Avicel® PH102) were passed through a 30 mesh screen.

The sodium croscarmellose (FMC Ac-Di-Sol®), SLS, Intermediate F, and lactose (Foremost FastFlo® Lactose #316) were also passed, individually in the preceding order, through the same 30 mesh screen. A nitrogen purge was used when screening Intermediate F. The screened components were loaded into a 10 cubic feet V-blender, which was purged with nitrogen, and blended for about 180 (+/−10) inversions.

The Magnesium Stearate was filtered through a 40 mesh screen sieve into the blending container and mixed to provide about 54 inversions.

The resulting mixture was compressed into tablets using a fully tooled 36 Fette 2090 press with 0.568″×0.2885″ caplet type B tooling set to produce a tablet having an initial target hardness of about 10 Kp ±20%.

### Example 9

### Exemplary Tablet 10 (Tablet 9 with Spray-Coating)

A batch of caplet-shaped tablets from Example 8 was spray-coated with OPADRY® II (Blue, Colorcon) to a weight gain of about 3.0% using a 24″ coating pan configured with the parameters in Table 1-9 followed by wax coating and then printing using Opacode® S-1-17823 (Solvent based Black, Colorcon).

TABLE 1-9
Spray-Coating Process Parameters

| Coating Parameters 24″ Pan | Target |
|---|---|
| Pan Load (kg) | 14 |
| Inlet Temperature (° C.) * | * |
| Pan Speed (rpm) | 10 |
| Jog Time (sec) | |
| # of Spray Guns | 2 |
| Solids Content (% w/w) | 20 |
| Gun to Bed Distance (inches) | 6 |
| Inlet Air Flow (cfm) | 300 |
| Spray Rate (g/min) | 35 |
| Exhaust Temperature (° C.) | 50 |
| Atomization Pressure (psi) | 42 |

* Inlet temperature is monitored to achieve target exhaust temperature. Initial inlet temperature should be set at about 75° C. to achieve target exhaust temp.

The OPADRY® II suspension was prepared by measuring an amount of de-ionized water which when combined with OPADRY® II would produce a total solids content of 20% w/w. The water is mixed to a vortex followed by addition of OPADRY® II over a period of approximately 5 minutes. Once the OPADRY® II powder was wetted, mixing was continued to ensure that all solid material is well-dispersed. The suspension is then charged into a Thomas 24″ pan coating instrument using coating conditions outlined in Table 1-9.

Uncoated tablets are placed into the coating pan and pre-warmed. The inlet was increased from room temperature to about 55° C. and then increased as necessary to provide the exhaust temperature in Table 1-9. The coating process was performed with 20% w/w OPADRY® II (85 Series Blue) coating dispersion to obtain a target weight gain of about 3%. The coated tablets were then allowed to tumble for about 2 minutes without spraying. The bed temperature was then allowed to cool to about 35° C.

Upon cooling, the Carnauba wax powder was weighed out in the amount of about 0.01% w/w of the starting tablet core weight. With the air flow off, the carnauba wax powder was sprinkled evenly on the tablet bed. The pan bed was turned on to the speed indicated in Table 1-9. After 5 minutes, the air flow was turned on (without heating) to the setting indicated in Table 1-9. After about one minute, the air flow and pan were turned off.

Once coated with OPADRY® II, the tablets are then labeled using a Hartnett Delta tablet printer charged with Opacode® S-1-17823.

### Example 10

### Exemplary Tablet 11 (Formulated with HPMCAS Polymer to have 150 mg of Compound 1)

A batch of caplet-shaped tablets was formulated to have about 150 mg of Compound 1 per tablet using the amounts of ingredients recited in Table 1-10.

TABLE 1-10
Ingredients for Exemplary Tablet 11.

| | Percent Dose % | Dose | Batch |
|---|---|---|---|
| Tablet Formulation | wt./wt. | (mg) | (g) |
| Intermediate F | 34.09% | 187.5 | 23.86 |
| Microcrystalline cellulose | 30.51% | 167.8 | 21.36 |
| Lactose | 30.40% | 167.2 | 21.28 |
| Sodium croscarmellose | 3.000% | 16.50 | 2.100 |
| SLS | 0.500% | 2.750 | 0.3500 |
| Colloidal silicon dioxide | 0.500% | 2.750 | 0.3500 |
| Magnesium stearate | 1.000% | 5.500 | 0.7000 |
| Total | 100% | 550 | 70 |

The colloidal silicon dioxide (Cabot Cab-O-Sil® M-5P Fumed Silicon Dioxide) and the microcrystalline cellulose (FMC MCC Avicel® PH102) were passed through a 30 mesh screen.

The sodium croscarmellose (FMC Ac-Di-Sol®), SLS, Intermediate F, and lactose (Foremost FastFlo® Lactose #316) were also passed, individually in the preceding order, through the same 30 mesh screen. A nitrogen purge was used when screening Intermediate F. The screened components were loaded into a 10 cubic feet V-blender, which was purged with nitrogen, and blended for about 180 (+/−10) inversions.

The Magnesium Stearate was filtered through a 40 mesh screen sieve into the blending container and mixed to provide about 54 inversions.

The resulting mixture was compressed into tablets using a fully tooled 36 Fette 2090 press with 0.568″×0.2885″ caplet type B tooling set to produce a tablet having an initial target hardness of about 10 Kp±20%.

### Example 11

### Exemplary Tablet 12 (Tablet 11 with Spray-Coating)

A batch of caplet-shaped tablets from Example 10 was spray-coated with OPADRY® II (Blue, Colorcon) to a weight gain of about 3.0% using a 24″ coating pan configured with the parameters in Table 1-11 followed by wax coating and then printing using Opacode® S-1-17823 (Solvent based Black, Colorcon).

TABLE 1-11
Spray-Coating Process Parameters

| Coating Parameters 24″ Pan | Target |
|---|---|
| Pan Load (kg) | 14 |
| Inlet Temperature (° C.) * | * |
| Pan Speed (rpm) | 10 |
| Jog Time (sec) | 2-5 sec every |
| | 60 sec |
| # of Spray Guns | 2 |
| Solids Content (% w/w) | 20 |
| Gun to Bed Distance (inches) | 6 |
| Inlet Air Flow (cfm) | 300 |
| Spray Rate (g/min) | 35 |
| Exhaust Temperature (° C.) | 50 |
| Atomization Pressure (psi) | 42 |

* Inlet temperature is monitored to achieve target exhaust temperature. Initial inlet temperature should be set at about 75° C. to achieve target exhaust temp.

The OPADRY® II suspension was prepared by measuring an amount of de-ionized water which when combined with OPADRY® II would produce a total solids content of 20% w/w. The water is mixed to a vortex followed by addition of OPADRY® II over a period of approximately 5 minutes. Once the OPADRY® II powder was wetted, mixing was continued to ensure that all solid material is well-dispersed. The suspension is then charged into a Thomas 24″ pan coating instrument using coating conditions outlined in Table 1-11.

Uncoated tablets are placed into the coating pan and pre-warmed. The inlet was increased from room temperature to about 55° C. and then increased as necessary to provide the exhaust temperature in Table 1-11. The coating process was performed with 20% w/w OPADRY® II (85 Series Blue) coating dispersion to obtain a target weight gain of about 3%. The coated tablets were then allowed to tumble for about 2 minutes without spraying. The bed temperature was then allowed to cool to about 35° C.

Upon cooling, the Carnauba wax powder was weighed out in the amount of about 0.01% w/w of the starting tablet core weight. With the air flow off, the carnauba wax powder was sprinkled evenly on the tablet bed. The pan bed was turned on to the speed indicated in Table 1-11. After 5 minutes, the air flow was turned on (without heating) to the setting indicated in Table 1-11. After about one minute, the air flow and pan were turned off.

Once coated with OPADRY® II, the tablets are then labeled using a Hartnett Delta tablet printer charged with Opacode® S-1-17823.

### Example 12

### Exemplary Tablet 13 (Formulated with HPMCAS Polymer to have 150 mg of Compound 1)

A batch of caplet-shaped tablets is formulated to have about 150 mg of Compound 1 per tablet using the amounts of ingredients recited in Table 1-12.

TABLE 1-12
Ingredients for Exemplary Tablet 13.

| | Percent Dose |
|---|---|
| Tablet Formulation | % Wt./Wt. |
| Intermediate H | 34.1% |
| Microcrystalline cellulose | 30.5% |
| Lactose | 30.4% |
| Sodium croscarmellose | 3.000% |
| SLS | 0.500% |
| Colloidal silicon dioxide | 0.500% |
| Magnesium stearate | 1.000% |
| Total | 100% |

The colloidal silicon dioxide (Cabot Cab-O-Sil® M-5P Fumed Silicon Dioxide) and the microcrystalline cellulose (FMC MCC Avicel® PH102) are passed through a 30 mesh screen.

The sodium croscarmellose (FMC Ac-Di-Sol®), SLS, Intermediate H, and lactose (Foremost FastFlo® Lactose #316) are also passed, individually in the preceding order, through the same 30 mesh screen. A nitrogen purge is used when screening Intermediate H. The screened components are loaded into a 10 cubic feet V-blender, which is purged with nitrogen, and blended for about 180 (+/−10) inversions.

The Magnesium Stearate is filtered through a 40 mesh screen sieve into the blending container and mixed to provide about 54 inversions.

The resulting mixture is compressed into tablets using a fully tooled 36 Fette 2090 press with 0.568″×0.2885″ caplet type B tooling set to produce a tablet having an initial target hardness of about 10 Kp±20%.

### Example 13

### Exemplary Tablet 14 (Tablet 13 with Spray-Coating)

A batch of caplet-shaped tablets from Example 12 is spray-coated with OPADRY® II (Blue, Colorcon) to a weight gain of about 3.0% using a Thomas 48″ coating pan configured with the parameters in Table 1-13 followed by wax coating and then printing using Opacode® S-1-17823 (Solvent based Black, Colorcon).

TABLE 1-13
Spray-Coating Process Parameters

| Coating Parameters 48″ Pan | Target |
|---|---|
| Pan Load (kg) | up to 120 |
| Inlet Temperature (° C.) * | * |
| # of Spray Guns | 4 |
| Solids Content (% w/w) | 20 |
| Gun to Bed Distance (inches) | 7-7.5 |
| Inlet Air Flow (cfm) | 1050-2400 |
| Spray Rate (ml/min) | 203-290 |
| Exhaust Temperature (° C.) | 40-65 |
| Atomization Pressure (slpm) | 145 |

* Inlet temperature is monitored to achieve target exhaust temperature. Initial inlet temperature should be set at about 50-75° C. to achieve target exhaust temp.

The OPADRY® II suspension is prepared by measuring an amount of de-ionized water which when combined with OPADRY® II would produce a total solids content of 20 % w/w. The water is mixed to a vortex followed by addition of OPADRY® II over a period of approximately 5 minutes. Once the OPADRY® II powder is wetted, mixing is continued to ensure that all solid material is well-dispersed. The suspension is then charged into a Thomas 48″ pan coating instrument using coating conditions outlined in Table 1-13. In other examples, the suspension can be coated with a Thomas 24″ pan coating instrument.

Uncoated tablets are placed into the coating pan and pre-warmed. The inlet is increased from room temperature to about 55° C. and then increased as necessary to provide the exhaust temperature in Table 1-13. The coating process is performed with 20% w/w OPADRY® II (85 Series Blue) coating dispersion to obtain a target weight gain of about 3%. The coated tablets are then allowed to tumble for about 2 minutes without spraying. The bed temperature is then allowed to cool to about 35° C.

Upon cooling, the Carnauba wax powder is weighed out in the amount of about 0.01% w/w of the starting tablet core weight. With the air flow off, the carnauba wax powder is sprinkled evenly on the tablet bed. The pan bed is turned on to the speed indicated in Table 1-13. After 5 minutes, the air flow is turned on (without heating) to the setting indicated in Table 1-13. After about one minute the air flow and pan is turned off.

Once coated with OPADRY® II, the tablets are then labeled using a Hartnett Delta tablet printer charged with Opacode® S-1-17823.

Another aspect of the present invention provides a method of producing a pharmaceutical composition comprising providing an admixture of a solid dispersion of substantially amorphous or amorphous Compound 1, a binder, a glidant, a surfactant, a lubricant, a disintegrant, and a filler, and compressing the admixture into a tablet having a dissolution of at least about 50% in about 30 minutes.

Each of the ingredients of this admixture is described above and in the Examples below. Furthermore, the admixture can comprise optional additives such as one or more colorants, one or more flavors, and/or one or more fragrances as described above and in the Examples below. And, the relative concentrations (e.g., wt %) of each of these ingredients (and any optional additives) in the admixture is also presented above and in the Examples below. The ingredients constituting the admixture can be provided sequentially or in any combination of additions; and, the ingredients or combination of ingredients can be provided in any order. In one embodiment, the lubricant is the last component added to the admixture.

In one embodiment, the admixture comprises a solid dispersion of substantially amorphous Compound 1, a binder, a glidant, a surfactant, a lubricant, a disintegrant, and a filler, wherein each of these ingredients is provided in a powder form (e.g., provided as particles having a mean diameter, measured by light scattering, of 250 μm or less (e.g., 150 μm or less, 100 μm or less, 50 μm or less, 45 μm or less, 40 μm or less, or 35 μm or less)). For instance, the admixture comprises a solid dispersion of amorphous Compound 1, a binder, a glidant, a surfactant, a lubricant, a disintegrant, and a filler, wherein each of these ingredients is provided in a powder form (e.g., provided as particles having a mean diameter, measured by light scattering, of 250 μm or less (e.g., 150 μm or less, 100 μm or less, 50 μm or less, 45 μm or less, 40 μm or less, or 35 μm or less)).

In another embodiment, the admixture comprises a solid dispersion of substantially amorphous Compound 1, a binder, a glidant, a surfactant, a lubricant, a disintegrant, and a filler, wherein each of these ingredients is substantially free of water. Each of the ingredients comprises less than 5 wt % (e.g., less than 2 wt %, less than 1 wt %, less than 0.75 wt %, less than 0.5 wt %, or less than 0.25 wt %) of water by weight of the ingredient. For instance, the admixture comprises a solid dispersion of amorphous Compound 1, a binder, a glidant, a surfactant, a lubricant, a disintegrant, and a filler, wherein each of these ingredients is substantially free of water. Each of the ingredients comprises less than 5 wt % (e.g., less than 2 wt %, less than 1 wt %, less than 0.75 wt %, less than 0.5 wt %, or less than 0.25 wt %) of water by weight of the ingredient.

In another embodiment, compressing the admixture into a tablet is accomplished by filling a form (e.g., a mold) with the admixture and applying pressure to admixture. This can be accomplished using a die press or other similar apparatus. It is also noted that the application of pressure to the admixture in the form can be repeated using the same pressure during each compression or using different pressures during the compressions. In another example, the admixture is compressed using a die press that applies sufficient pressure to form a tablet having a dissolution of about 50% or more at about 30 minutes (e.g., about 55% or more at about 30 minutes or about 60% or more at about 30 minutes). For instance, the admixture is compressed using a die press to produce a tablet hardness of at least about 5 Kp (at least about 5.5 Kp, at least about 6 Kp, at least about 7 Kp, at least about 11 Kp, or at least 21Kp). In some instances, the admixture is compressed to produce a tablet hardness of between about 6 and 21 Kp.

In some embodiments, tablets comprising a pharmaceutical composition as described herein can be coated with about 3.0 wt % of a film coating comprising a colorant by weight of the tablet. In certain instances, the colorant suspension or solution used to coat the tablets comprises about 20% w/w of solids by weight of the colorant suspension or solution. In still further instances, the coated tablets can be labeled with a logo, other image or text.

In another embodiment, the method of producing a pharmaceutical composition comprises providing an admixture of a solid dispersion of substantially amorphous Compound 1, a binder, a glidant, a surfactant, a lubricant, a disintegrant, and a filler; mixing the admixture until the admixture is substantially homogenous, and compressing the admixture into a tablet as described above or in the Examples below. Or, the method of producing a pharmaceutical composition comprises providing an admixture of a solid dispersion of amorphous Compound 1, a binder, a glidant, a surfactant, a lubricant, a disintegrant, and a filler; mixing the admixture until the admixture is substantially homogenous, and compressing the admixture into a tablet as described above or in the Examples below. For example, the admixture is mixed by stirring, blending, shaking, or the like using hand mixing, a mixer, a blender, any combination thereof, or the like. When ingredients or combinations of ingredients are added sequentially, mixing can occur between successive additions, continuously throughout the ingredient addition, after the addition of all of the ingredients or combinations of ingredients, or any combination thereof. The admixture is mixed until it has a substantially homogenous composition.

### IV.B.3. Administration of Compound 1 Tablet and SDD Formulation

Another aspect of the present invention provides a method of administering a pharmaceutical composition by orally administering to a patient at least once per day the composition comprising a solid dispersion of substantially amorphous or amorphous Compound 1, in which the solid dispersion comprises at least about 100 mg of substantially amorphous or amorphous Compound 1.

Another aspect of the present invention provides a method of administering a pharmaceutical composition by orally administering to a patient at least once per day the composition comprising a solid dispersion of substantially amorphous or amorphous Compound 1, in which the solid dispersion comprises at least about 150 mg of substantially amorphous or amorphous Compound 1.

Another aspect of the present invention provides a method of administering a pharmaceutical composition by orally administering to a patient twice per day the composition comprising a solid dispersion of substantially amorphous or amorphous Compound 1, in which the solid dispersion comprises at least about 100 mg of substantially amorphous or amorphous Compound 1.

Another aspect of the present invention provides a method of administering a pharmaceutical composition by orally administering to a patient twice per day the composition comprising a solid dispersion of substantially amorphous or amorphous Compound 1, in which the solid dispersion comprises at least about 150 mg of substantially amorphous or amorphous Compound 1.

Another aspect of the present invention provides a method of administering a pharmaceutical composition by orally administering to a patient once every 12 hours day. The composition comprising a solid dispersion of substantially amorphous or amorphous Compound 1, in which the solid dispersion comprises at least about 100 mg of substantially amorphous or amorphous Compound 1.

Another aspect of the present invention provides a method of administering a pharmaceutical composition by orally administering to a patient once every 12 hours. The composition comprising a solid dispersion of substantially amorphous or amorphous Compound 1, in which the solid dispersion comprises at least about 150 mg of substantially amorphous or amorphous Compound 1.

In still other aspects of the present invention, a pharmaceutical composition as described herein is orally administered to a patient once every 24 hours.

Another aspect of the present invention provides a method of administering a pharmaceutical composition by orally administering to a patient once per day the composition comprising a solid dispersion of substantially amorphous or amorphous Compound 1, in which the solid dispersion comprises at least about 100 mg of substantially amorphous or amorphous Compound 1.

Another aspect of the present invention provides a method of administering a pharmaceutical composition by orally administering to a patient once per day the composition comprising a solid dispersion of substantially amorphous or amorphous Compound 1, in which the solid dispersion comprises at least about 150 mg of substantially amorphous or amorphous Compound 1.

In some embodiments, the present invention provides a method of administering a pharmaceutical composition comprising orally administering to a patient at least one tablet comprising:
a. a solid dispersion comprising about 100 mg of substantially amorphous or amorphous Compound 1 and HPMCAS;
b. a filler;
c. a disintegrant;
d. a surfactant;
e. a binder;
f. a glidant; and
g. a lubricant.

In some embodiments, the present invention provides a method of administering a pharmaceutical composition comprising orally administering to a patient at least one tablet comprising:
a. a solid dispersion comprising about 150 mg of substantially amorphous or amorphous Compound 1 and HPMCAS;
b. a filler;
c. a disintegrant;
d. a surfactant;
e. a binder;
f. a glidant; and
g. a lubricant.

In some embodiments, the present invention provides for a method of orally administering the pharmaceutical composition described herein once a day. In other embodiments, the present invention provides for a method of orally administering the pharmaceutical composition described herein twice a day.

Another aspect of the present invention provides a method of administering a pharmaceutical composition by orally administering to a patient at least once per day at least one tablet comprising a solid dispersion of substantially amorphous or amorphous Compound 1, a filler, a binder, a glidant, a disintegrant, a surfactant, and a lubricant, in which the solid dispersion comprises at least about 100 mg of substantially amorphous or amorphous Compound 1. In some embodiments, the tablet is orally administered to the patient once per day. In another method, the administration comprises orally administering to a patient twice per day at least one tablet comprising a solid dispersion of substantially amorphous or amorphous Compound 1, a filler, a binder, a glidant, a disintegrant, a surfactant, and a lubricant, in which the solid dispersion contains at least about 100 mg of substantially amorphous or amorphous Compound 1. Other tablets useful in this method comprise a solid dispersion containing at least about 150 mg of substantially amorphous or amorphous Compound 1. In another method, the administration includes orally administering to a patient twice per day at least one tablet comprising a solid dispersion of substantially amorphous or amorphous Compound 1, a filler, a binder, a glidant, a disintegrant, a surfactant, and a lubricant, in which the solid dispersion contains at least about 150 mg of substantially amorphous or amorphous Compound 1.

In another embodiment, the method of administering a pharmaceutical composition includes orally administering to a patient once per day at least one tablet comprising a pharmaceutical composition containing a solid dispersion of Compound 1, a filler, a binder, a glidant, a disintegrant, a surfactant, and a lubricant, each of which is described above and in the Examples below, wherein the solid dispersion comprises at least about 100 mg, or at least about 150 mg) of substantially amorphous Compound 1 or amorphous Compound 1. For example, the method of administering a pharmaceutical composition includes orally administering to a patient once per day one tablet comprising a pharmaceutical composition containing a solid dispersion of Compound 1, a filler, a binder, a glidant, a disintegrant, a surfactant, and a lubricant, wherein the solid dispersion comprises at least 100 mg, or at least 150 mg of substantially amorphous Compound 1 or amorphous Compound 1.

In another embodiment, the method of administering a pharmaceutical composition includes orally administering to a patient twice per day one tablet comprising a pharmaceutical composition containing a solid dispersion of Compound 1, a filler, a binder, a glidant, a disintegrant, a surfactant, and a lubricant, wherein the solid dispersion comprises at least 100 mg or at least 150 mg of substantially amorphous Compound 1 or amorphous Compound 1.

In one embodiment, the method of administering a pharmaceutical composition includes orally administering to a patient a formulation comprising from about 25 mg to about 300 mg of Compound 1. In one embodiment, the method of administering a pharmaceutical composition includes orally administering to a patient one or more tablets, each tablet comprising about 100 mg, about 150 mg, or about 250 mg of Compound 1. In some embodiments, the method includes administering a tablet comprising about 250 mg of Compound 1. In some embodiments, the method includes administering a tablet comprising about 150 mg of Compound 1 and a tablet comprising about 100 mg of Compound 1. In one embodiment, the method includes administering to a patient a tablet comprising about 100 mg of Compound 1 as described in Example 8 or Example 9 of Section IV.B.2, entitled “Preparation of Compound 1 Tablet and SDD Formulation.” In another embodiment, the method includes administering to a patient a tablet comprising about 150 mg of Compound 1 as described in Example 10, Example 11, Example 12 or Example 13 of Section IV.B.2, entitled “Preparation of Compound 1 Tablet and SDD Formulation.” In a further embodiment, the method includes administering to a patient a tablet comprising about 100 mg of Compound 1 as described in Example 8 or Example 9 of Section IV.B.2, entitled “Preparation of Compound 1 Tablet and SDD Formulation” and a tablet comprising about 150 mg of Compound 1 as described in Example 10, Example 11, Example 12 or Example 13 of Section IV.B.2, entitled “Preparation of Compound 1 Tablet and SDD Formulation.” In some embodiments, the method includes administering the tablet comprising 100 mg of Compound 1 and the tablet comprising 150 mg of Compound 1 in the same vehicle. In some embodiments, the method includes administering the tablet comprising 100 mg of Compound 1 and the tablet comprising 150 mg of Compound 1 in separate vehicles.

It is noted that the methods of administration of the present invention can optionally include orally administering a beverage (water, milk, or the like), food, and/or additional pharmaceutical compositions including additional APIs. When the method of administration includes orally administering a beverage (water, milk, or the like), food (including a standard high fat high calorie CF meal or snack), and/or additional pharmaceutical compositions including additional APIs, the oral administration of the beverage, food, and/or additional API can occur concurrently with the oral administration of the tablet, prior to the oral administration of the tablet, and/or after the administration of the tablet. For instance, in one example, the method of administering a pharmaceutical composition includes orally administering to a patient at least once per day at least one tablet comprising a pharmaceutical composition containing a solid dispersion of substantially amorphous Compound 1 or amorphous Compound 1, a filler, a binder, a glidant, a disintegrant, a surfactant, a lubricant, and a second API. In still other examples, the method of administering a pharmaceutical composition includes orally administering to a patient every 12 hours at least one tablet comprising a pharmaceutical composition as described herein, in which the tablet is administered about 30 minutes after consuming a high fat, high calorie CF meal or snack.

### V. Methods of Using

In one aspect, the invention features a pharmaceutical composition comprising Compound 1. In some embodiments of this aspect, Compound 1 is Compound 1 Form C. In some further embodiments of this aspect, the composition comprises Compound 1 First Formulation. In some other embodiments, the composition comprises Compound 1 SDD and Tablet Formulation.

In still another embodiment, the formulation comprises an additional agent. In one embodiment, the additional agent is selected from a mucolytic agent, bronchodialator, an anti-biotic, an anti-infective agent, an anti-inflammatory agent, a nutritional agent or a CFTR modulator other than Compound 1.

In one embodiment, the additional agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In another embodiment, the additional agent is a mucolyte. Exemplary mucolytes useful herein includes Pulmozyme®.

In another embodiment, the additional agent is a bronchodialator. Exemplary bronchodilators include albuterol, metaprotenerol sulfate, pirbuterol acetate, salmeterol, or tetrabuline sulfate.

In another embodiment, the additional agent is effective in restoring lung airway surface liquid. Such agents improve the movement of salt in and out of cells, allowing mucus in the lung airway to be more hydrated and, therefore, cleared more easily. Exemplary such agents include hypertonic saline, denufosol tetrasodium ([[(3S,5R)-5-(4-amino-2-oxopyrimidin-1-yl)-3 -hydroxyoxolan-2-yl]methoxy-hydroxyphosphoryl] [[[(2R,3S,4R,5R)-5-(2,4-dioxopyrimidin-1-yl)-3,4-dihydroxyoxolan-2-yl]methoxy-hydroxyphosphoryl]oxy-hydroxyphosphoryl]hydrogen phosphate), or bronchitol (inhaled formulation of mannitol).

In another embodiment, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In another embodiment, the additional agent is a CFTR modulator other than compound 1, i.e., an agent that has the effect of modulating CFTR activity. Exemplary such agents include ataluren (“PTC1240”; 345-(2-fluorophenyl)-1,2,4-oxadiazol-3-yl]benzoic acid), sinapultide, lancovutide, depelestat (a human recombinant neutrophil elastase inhibitor), cobiprostone (7-{(2R,4aR,5R,7aR)-2-[(3S)-1,1-difluoro-3-methylpentyl]-2-hydroxy-6-oxooctahydrocyclopenta[b]pyran-5-yl}heptanoic acid), or (3-(6-(1-(2,2-difluorobenzo[d][1,3]dioxo1-5-yl) cyclopropanecarboxamido)-3-methylpyridin-2-yl)benzoic acid. In another embodiment, the additional agent is (3-(6-(1-(2,2-difluorobenzo[d][1,3]dioxol-5-yl)cyclopropanecarboxamido)-3-methylpyridin-2-yl)benzoic acid.

In another embodiment, the additional agent is a nutritional agent. Exemplary such agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In one aspect, the present invention features a method of treating a CFTR mediated disease in a human comprising administering to the human an effective amount of a pharmaceutical formulation comprising Compound 1 as described herein.

In another aspect, the invention also provides a method of treating or lessening the severity of a disease in a patient comprising administering to said patient one of the pharmaceutical compositions as defined herein, and said disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulinemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Pelizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebellar ataxia type I, spinal and bulbar muscular atrophy, dentatorubral pallidoluysian, and myotonic dystrophy, as well as spongiform encephalopathies, such as hereditary Creutzfeldt-Jakob disease (due to prion protein processing defect), Fabry disease, Gerstmann-Sträussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

In some embodiments, the method includes treating or lessening the severity of cystic fibrosis in a patient comprising administering to said patient one of the pharmaceutical compositions as defined herein. In certain embodiments, the patient possesses mutant forms of human CFTR. In other embodiments, the patient possesses one or more of the following mutations possessing one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G.

In one aspect, the method includes treating or lessening the severity of Cystic Fibrosis in a patient by administering to said patient Compound 1 or one of the compositions as defined herein, wherein the patient possesses the ΔF508 mutation of human CFTR and one or more human CFTR mutations selected from G178R, G551S, G970R, G1244E, S1255P, G1349D, S549N, S549R, S1251N, E193K, F1052V, G1069R, R117C, D11011, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G.

In one aspect, the method includes treating or lessening the severity of Cystic Fibrosis in a patient by administering to said patient Compound 1 or one of the compositions as defined herein, wherein the patient possesses the G551D mutation of human CFTR and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G.

In one aspect, the method includes treating or lessening the severity of Cystic Fibrosis in a patient by administering to said patient Compound 1 or one of the compositions as defined herein, wherein the patient possesses the ΔF508 mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G on at least one allele.

In one aspect, the method includes treating or lessening the severity of Cystic Fibrosis in a patient by administering to said patient Compound 1 or one of the compositions as defined herein, wherein the patient possesses the G551D mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G on at least one allele.

In one aspect, the method includes treating or lessening the severity of Cystic Fibrosis in a patient by administering to said patient Compound 1 or one of the compositions as defined herein, wherein the patient possesses the ΔF508 mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G on at least one allele.

In one aspect, the method includes treating or lessening the severity of Cystic Fibrosis in a patient by administering to said patient Compound 1 or one of the compositions as defined herein, wherein the patient possesses the G551 D mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G on at least one allele.

In one aspect, the method includes treating or lessening the severity of Cystic Fibrosis in a patient by administering to said patient Compound 1 or one of the compositions as defined herein, wherein the patient possesses the R117H mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G on at least one allele.

In one aspect, the method includes treating or lessening the severity of Cystic Fibrosis in a patient by administering to said patient Compound 1 or one of the compositions as defined herein, wherein the patient possesses the R117H mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1 G->C, 1898+5G->T, 3850−3T->G, IVS14b+5G->A, 1898+1G->T, 4005+2T->C and 621+3A->G on at least one allele.

In some embodiments, the method includes treating or lessening the severity of cystic fibrosis in a patient comprising administering to said patient one of the pharmaceutical compositions as defined herein. In certain embodiments, the patient possesses mutant forms of human CFTR. In other embodiments, the patient possesses one or more of the following mutations possessing one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H.

In one aspect, the method includes treating or lessening the severity of Cystic Fibrosis in a patient by administering to said patient Compound 1 or one of the compositions as defined herein, wherein the patient possesses the ΔF508 mutation of human CFTR and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H.

In one aspect, the method includes treating or lessening the severity of Cystic Fibrosis in a patient by administering to said patient Compound 1 or one of the compositions as defined herein, wherein the patient possesses the G551D mutation of human CFTR and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H.

In one aspect, the method includes treating or lessening the severity of Cystic Fibrosis in a patient by administering to said patient Compound 1 or one of the compositions as defined herein, wherein the patient possesses the ΔF508 mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In one aspect, the method includes treating or lessening the severity of Cystic Fibrosis in a patient by administering to said patient Compound 1 or one of the compositions as defined herein, wherein the patient possesses the G551D mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In one aspect, the method includes treating or lessening the severity of Cystic Fibrosis in a patient by administering to said patient Compound 1 or one of the compositions as defined herein, wherein the patient possesses the ΔF508 mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In one aspect, the method includes treating or lessening the severity of Cystic Fibrosis in a patient by administering to said patient Compound 1 or one of the compositions as defined herein, wherein the patient possesses the G551D mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In one aspect, the method includes treating or lessening the severity of Cystic Fibrosis in a patient by administering to said patient Compound 1 or one of the compositions as defined herein, wherein the patient possesses the R117H mutation of human CFTR on at least one allele and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In one aspect, the method includes treating or lessening the severity of Cystic Fibrosis in a patient by administering to said patient Compound 1 or one of the compositions as defined herein, wherein the patient possesses the R117H mutation of human CFTR on both alleles and one or more human CFTR mutations selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N and D1152H on at least one allele.

In some embodiments of any of the above aspects, the human CFTR mutation is selected from G178R, G551S, G970R, G1244E, S1255P, G1349D, S549N, S549R, S1251N, E193K, F1052V and G1069R. In some embodiments of any of the above aspects, the human CFTR mutation is selected from G178R, G551S, G970R, G1244E, S1255P, G1349D, S549N, S549R and S1251N. In some embodiments of any of the above aspects, the human CFTR mutation is selected from E193K, F1052V and G1069R. In some embodiments of the above aspects, the method produces a greater than 10-fold increase in chloride transport relative to baseline chloride transport.

In some embodiments of any of the above aspects, the human CFTR mutation is selected from R117C, D110H, R347H, R352Q, E56K, P67L, L206W, A455E, D579G, S1235R, S945L, R1070W, F1074L, D110E, D1270N and D1152H. In some embodiments of the above aspects, the method produces an increase in chloride transport which is greater or equal to 10% above the baseline chloride transport.

In some embodiments of any of the above aspects, the human CFTR mutation is selected from 1717−1G->A, 621+1G->T, 3120+1G->A, 1898+1G->A, 711+1G->T, 2622+1G->A, 405+1G->A, 406−1G->A, 4005+1G->A, 1812−1G->A, 1525−1G->A, 712−1G->T, 1248+1G->A, 1341+1G->A, 3121−1G->A, 4374+1G->T, 3850−1G->A, 2789+5G->A, 3849+10 kbC->T, 3272−26A->G, 711+5G->A, 3120G->A, 1811+1.6 kbA->G, 711+3A->G, 1898+3A->G, 1717−8G->A, 1342−2A->C, 405+3A->C, 1716G/A, 1811+1G->C, 1898+5G->T, 3850−3T->G, IVS14b+5G->A, 1898+1G->T, 4005+2T->C and 621+3A->G. In some embodiments of any of any of the above aspects, the human CFTR mutation is selected from CFTR mutation selected from 1717−1G->A, 1811+1.6 kbA->G, 2789+5G->A, 3272−26A->G and 3849+10 kbC->T. In some further embodiments of any of the above aspects, the human CFTR mutation is selected from CFTR mutation selected from 2789+5G->A and 3272−26A->G.

In certain embodiments, the method of treating or lessening the severity of Osteoporosis in a patient comprises administering to said patient a pharmaceutical composition as described herein.

In certain embodiments, the method of treating or lessening the severity of Osteopenia in a patient comprises administering to said patient a pharmaceutical composition as described herein.

In certain embodiments, the method of bone healing and/or bone repair in a patient comprises administering to said patient a pharmaceutical composition as described herein.

In certain embodiments, the method of reducing bone resorption in a patient comprises administering to said patient a pharmaceutical composition as described herein.

In certain embodiments, the method of increasing bone deposition in a patient comprises administering to said patient a pharmaceutical composition as described herein.

In certain embodiments, the method of treating or lessening the severity of COPD in a patient comprises administering to said patient a pharmaceutical composition as described herein.

In certain embodiments, the method of treating or lessening the severity of smoke induced COPD in a patient comprises administering to said patient a pharmaceutical composition as described herein.

In certain embodiments, the method of treating or lessening the severity of chronic bronchitis in a patient comprises administering to said patient a pharmaceutical composition as described herein.

In one aspect, the present invention features a kit comprising Compound 1. In one embodiment, the kit comprises Compound 1 and instructions for use thereof. In another embodiment, the kit comprises Compound 1 Form C. In another embodiment, the kit comprises Compound 1 First Formulation. In another embodiment, the kit comprises Compound 1 Tablet and SDD Formulation.

### VI. Assays

### VI.1. Protocol for Detecting and Measuring ΔF508-CFTR Potentiation Properties of Compounds

### Membrane Potential Optical Methods for Assaying ΔF508-CFTR Modulation Properties of Compounds,

The assay utilizes fluorescent voltage sensing dyes to measure changes in membrane potential using a fluorescent plate reader (e.g., FLIPR III, Molecular Devices, Inc.) as a readout for increase in functional ΔF508-CFTR in NIH 3T3 cells. The driving force for the response is the creation of a chloride ion gradient in conjunction with channel activation by a single liquid addition step after the cells have previously been treated with compounds and subsequently loaded with a voltage sensing dye.

### Identification of Potentiator Compounds

To identify potentiators of ΔF508-CFTR, a double-addition HTS assay format was developed. This HTS assay utilizes fluorescent voltage sensing dyes to measure changes in membrane potential on the FLIPR III as a measurement for increase in gating (conductance) of ΔF508 CFTR in temperature-corrected ΔF508 CFTR NIH 3T3 cells. The driving force for the response is a Cl⁻ ion gradient in conjunction with channel activation with forskolin in a single liquid addition step using a fluorescent plate reader such as FLIPR III after the cells have previously been treated with potentiator compounds (or DMSO vehicle control) and subsequently loaded with a redistribution dye.

### Solutions

Bath Solution #1: (in mM) NaCl 160, KCl 4.5, CaCl₂ 2, MgCl₂ 1, HEPES 10, pH 7.4 with NaOH.

Chloride-free bath solution: Chloride salts in Bath Solution #1 (above) are substituted with gluconate salts.

### Cell Culture

NIH3T3 mouse fibroblasts stably expressing ΔF508-CFTR are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For all optical assays, the cells were seeded at ˜20,000/well in 384-well matrigel-coated plates and cultured for 2 hrs at 37° C. before culturing at 27° C. for 24 hrs. for the potentiator assay. For the correction assays, the cells are cultured at 27° C. or 37° C. with and without compounds for 16-24 hours. Electrophysiological Assays for assaying ΔF508-CFTR modulation properties of compounds.

### Ussing Chamber Assay

Ussing chamber experiments were performed on polarized airway epithelial cells expressing ΔF508-CFTR to further characterize the ΔF508-CFTR modulators identified in the optical assays. Non-CF and CF airway epithelia were isolated from bronchial tissue, cultured as previously described (Galietta, L. J. V., Lantero, S., Gazzolo, A., Sacco, O., Romano, L., Rossi, G. A., & Zegarra-Moran, O. (1998) In Vitro Cell. Dev. Biol. 34, 478-481), and plated onto Costar® Snapwell™ filters that were precoated with NIH3T3-conditioned media. After four days the apical media was removed and the cells were grown at an air liquid interface for >14 days prior to use. This resulted in a monolayer of fully differentiated columnar cells that were ciliated, features that are characteristic of airway epithelia. Non-CF HBE were isolated from non-smokers that did not have any known lung disease. CF-HBE were isolated from patients homozygous for ΔF508-CFTR.

HBE grown on Costar® Snapwell™ cell culture inserts were mounted in an Using chamber (Physiologic Instruments, Inc., San Diego, Calif.), and the transepithelial resistance and short-circuit current in the presence of a basolateral to apical Cl⁻ gradient (I_{SC}) were measured using a voltage-clamp system (Department of Bioengineering, University of Iowa, Iowa). Briefly, HBE were examined under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂, 10 Glucose, 10 HEPES (pH adjusted to 7.35 with NaOH) and the apical solution contained (in mM) 145 NaGluconate, 1.2 MgCl₂, 1.2 CaCl₂, 10 glucose, 10 HEPES (pH adjusted to 7.35 with NaOH).

### Identification of Potentiator Compounds

Typical protocol utilized a basolateral to apical membrane Cl⁻ concentration gradient. To set up this gradient, normal ringers was used on the basolateral membrane, whereas apical NaCl was replaced by equimolar sodium gluconate (titrated to pH 7.4 with NaOH) to give a large Cl⁻ concentration gradient across the epithelium. Forskolin (10 μM) and all test compounds were added to the apical side of the cell culture inserts. The efficacy of the putative ΔF508-CFTR potentiators was compared to that of the known potentiator, genistein.

### Patch-Clamp Recordings

Total Cl⁻ current in ΔF508-NIH3T3 cells was monitored using the perforated-patch recording configuration as previously described (Rae, J., Cooper, K., Gates, P., & Watsky, M. (1991) J. Neurosci. Methods 37, 15-26). Voltage-clamp recordings were performed at 22° C. using an Axopatch 200B patch-clamp amplifier (Axon Instruments Inc., Foster City, Calif.). The pipette solution contained (in mM) 150 N-methyl-D-glucamine (NMDG)-Cl, 2 MgCl₂, 2 CaCl₂, 10 EGTA, 10 HEPES, and 240 μg/mL amphotericin-B (pH adjusted to 7.35 with HCl). The extracellular medium contained (in mM) 150 NMDG-Cl, 2 MgCl₂, 2 CaCl₂, 10 HEPES (pH adjusted to 7.35 with HCl). Pulse generation, data acquisition, and analysis were performed using a PC equipped with a Digidata 1320 A/D interface in conjunction with Clampex 8 (Axon Instruments Inc.). To activate ΔF508-CFTR, 10 μM forskolin and 20 μM genistein were added to the bath and the current-voltage relation was monitored every 30 sec.

### Identification of Potentiator Compounds

The ability of ΔF508-CFTR potentiators to increase the macroscopic ΔF508-CFTR Cl⁻ current (I_{ΔF508}) in NIH3T3 cells stably expressing ΔF508-CFTR was also investigated using perforated-patch-recording techniques. The potentiators identified from the optical assays evoked a dose-dependent increase in IΔ_{F508} with similar potency and efficacy observed in the optical assays. In all cells examined, the reversal potential before and during potentiator application was around −30 mV, which is the calculated E_{Cl} (−28 mV).

### Cell Culture

NIH3T3 mouse fibroblasts stably expressing ΔF508-CFTR are used for whole-cell recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use to test the activity of potentiators; and incubated with or without the correction compound at 37° C. for measuring the activity of correctors.

### Single-Channel Recordings

Gating activity of wt-CFTR and temperature-corrected ΔF508-CFTR expressed in NIH3T3 cells was observed using excised inside-out membrane patch recordings as previously described (Dalemans, W., Barbry, P., Champigny, G., Jallat, S., Dott, K., Dreyer, D., Crystal, R. G., Pavirani, A., Lecocq, J-P., Lazdunski, M. (1991) Nature 354, 526-528) using an Axopatch 200B patch-clamp amplifier (Axon Instruments Inc.). The pipette contained (in mM): 150 NMDG, 150 aspartic acid, 5 CaCl₂, 2 MgCl₂, and 10 HEPES (pH adjusted to 7.35 with Tris base). The bath contained (in mM): 150 NMDG-Cl, 2 MgCl₂, 5 EGTA, 10 TES, and 14 Tris base (pH adjusted to 7.35 with HCl). After excision, both wt- and ΔF508-CFTR were activated by adding 1 mM Mg-ATP, 75 nM of the catalytic subunit of cAMP-dependent protein kinase (PKA; Promega Corp. Madison, Wis.), and 10 mM NaF to inhibit protein phosphatases, which prevented current rundown. The pipette potential was maintained at 80 mV. Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Pₒ) were determined from 120 sec of channel activity. The Pₒ was determined using the Bio-Patch software or from the relationship Pₒ=I/i(N), where I=mean current, i=single-channel current amplitude, and N=number of active channels in patch.

### Cell Culture

NIH3T3 mouse fibroblasts stably expressing ΔF508-CFTR are used for excised-membrane patch-clamp recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For single channel recordings, 2,500-5,000 cells were seeded on poly-L-lysine-coated glass coverslips and cultured for 24-48 hrs at 27° C. before use.

### Activity of the Compound 1

Compounds of the invention are useful as modulators of ATP binding cassette transporters. Table 1-14 below illustrates the EC50 and relative efficacy of Compound 1. In Table 1-14 below, the following meanings apply. EC50: “+++” means <10 uM; “++” means between 10 uM to 25 uM; “+” means between 25 uM to 60uM. % Efficacy: “+” means <25%; “++” means between 25% to 100%; “+++” means >100%.

TABLE 1-14

| Cmpd # | EC50 (μM) | % Activity |
|---|---|---|
| 1 | +++ | ++ |

### VI.2. Protocol for Detecting and Measuring CFTR Potentiation Properties of Compound 1 Against Various Human CFTR Mutations

### Generation of Recombinant Cell Lines Expressing Different CFTR Mutant Forms

DNA cloning: Wild-type CFTR coding region was inserted into pcDNA5/FRT (Invitrogen, San Diego, Calif.) between EcoRV and ApaI.

### Mutagenesis

Single CFTR gene mutations were introduced into the wild-type CFTR coding sequence by using QuickChange XL site-directed mutagenesis kit (Stratagene, Cambridge, UK). The CFTR coding region as well as its promoter sequence and 3′ untranslated sequence was fully sequenced to confirm the mutagenesis reaction.

### Cell Line Generation

The CFTR gene was stably expressed in Fisher rat thyroid (FRT) cells through FlpIn system. The FRT-FlpIn host cell line was generated by stably transfecting FRT cells with pFRT/lacZeo. The single integration of a FRT site was confirmed by Southern blot. After the mutant CFTR DNA was transfected into the FRT-FlpIn host cell line, the cells were incubate at 37° C. in Coon's modified Ham's F12 containing 10% FBS, 1% Pen/Strep, and 36 ml of Na-Bicarbonate for up to 8 passages under hygromycin selection (200 ug/ml).

Culture of Human Bronchial Epithelia (HBE) Isolated from CF Patients

Whole lungs were provided by the National Disease Research Interchange (Philadelphia, Pa.) through an agreement with the Cystic Fibrosis Foundation Therapeutics Incorporated and were obtained from non-CF or CF subjects following autopsy or lung transplantation. After removal, the intact lung was packed in ice cold PBS and processed within 24 hours. Non-CF and CF airway epithelia were isolated from bronchial tissue and cultured on 0.4 μm SnapWell™ culture inserts (Corning Catalog #3801) previously coated with NIH-3T3 conditioned media at a density of 5e5 cells/insert as previously described (2) with the following modifications; 1) Accutase (Innovative Cell Technologies Inc. San Diego, Calif.) was used to dissociate the cells, 2) all plastic culture ware and the Costar® Snapwell™ filters were pre-coated with NIH-3T3-conditioned media, and 3) bovine brain extract (LONZA; Kit #CC-4133, component #CC-4092C) was added to the differentiation media. After four days the apical media was removed and the cells were grown at an air liquid interface for >14 days prior to use. This resulted in a monolayer of fully differentiated columnar cells that were ciliated.

### Ussing Chamber Recordings

All cells were grown on Costar® Snapwell™ cell culture inserts at maintained at 37° C. prior to recording. The cell culture inserts were mounted into an Ussing chamber (VCC MC8; Physiologic Instruments, Inc., San Diego, Calif.) to record ISC in the voltage-clamp mode (Vhold=0 mV). For FRT cells, the basolateral membrane was permeabilized with 360 μg/ml Nystatin and a basolateral to apical Cl— gradient was established. The basolateral bath solution contained (in mM); 135 NaCl, 1.2 CaCl₂, 1.2 MgCl₂, 2.4 K₂HPO₄, 0.6 KHPO₄, 10 N-2-hydroxyethylpiperazine-N′-2-ethanesulfonic acid (HEPES), and 10 dextrose (titrated to pH 7.4 with NaOH). The apical NaCl was replaced by equimolar Na+gluconate (titrated to pH 7.4 with NaOH). For HBE cells, the ISC was measured in the presence of a basolateral to apical Cl— gradient. The normal Cl— solution contained (in mM) 145 NaCl, 3.3 K₂HPO₄, 1.2 MgCl₂, 1.2 CaCl₂, 10 Glucose, 10 HEPES (pH adjusted to 7.35 with NaOH) and the low Cl— solution contained (in mM) 145 NaGluconate, 1.2 MgCl₂, 1.2 CaCl₂, 10 glucose, 10 HEPES (pH adjusted to 7.35 with NaOH). All recordings were digitally acquired using a Acquire and Analyze software (version 2; Physiologic Instruments, Inc. San Diego, Calif.).

The 10 μM forskolin stimulated response in FRT cell expressing different mutant CFTR forms or in HBE cells isolated from CF patients was normalized to the 10 μM forskolin-stimulated response in FRT cells expressing wild-type CFTR or in HBE isolated from non-CF individuals and expressed as % wild-type CFTR. In HBE, amiloride was added prior to forskolin application to block the epithelial Na+ channel.

Using the FRT cell assay methods as described herein, Compound 1 produced a greater than 10-fold increase in chloride transport, relative to baseline chloride transport, in the human CFTR mutants G178R, G551S, G970R, G1244E, S1255P, G1349D, S549N, S549R and S1251N.

Using the FRT cell assay methods as described herein, Compound 1 produced an increase in chloride transport of greater than or equal to 10%, relative to baseline chloride transport, in the human CFTR mutants R117C, D110H, R347H, R352Q, E56K, P67L, L206W, A455E, D579G, S1235R, S945L, R1070W, F1074L, D110E, D1270N and D1152H.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method of treating a CFTR mediated disease in a human comprising administering Compound 1 to a patient possessing one or more human CFTR mutations selected from G178R, G551S, G970R, G1244E, S1255P, G1349D, S549N, S549R, S1251N, E193K, F1052V and G1069R.

2. The method of claim 1, wherein the one or more human CFTR mutations are selected from G178R, G551S, G970R, G1244E, S1255P, G1349D, S549N, S549R and S1251N.

3. The method of claim 1, wherein the one or more human CFTR mutations are selected from E193K, F1052V and G1069R.

4. A method of treating a CFTR mediated disease in a human comprising administering Compound 1 to a patient possessing one or more human CFTR mutations selected from R117C, D110H, R347H, R352Q, E56K, P67L, L206W, A455E, D579G, S1235R, S945L, R1070W, F1074L, D110E, D1270N and D1152H.

5. The method of claim 1, wherein the human also possesses one or more human CFTR mutations selected from ΔF508, R117H, and G551D.

6. The method of claim 2, wherein the human also possesses one or more human CFTR mutations selected from ΔF508, R117H, and G551D.

7. The method of claim 3, wherein the human also possesses one or more human CFTR mutations selected from ΔF508, R117H, and G551D.

8. The method of claim 4, wherein the human also possesses one or more human CFTR mutations selected from ΔF508, R117H, and G551D.

9. The method of claim 1, wherein Compound 1 is administered to a patient possessing one human CFTR mutation selected from G178R, G551S, G970R, G1244E, S1255P, G1349D, S549N, S549R, S1251N, E193K, F1052V and G1069R.

10. The method of claim 9, wherein Compound 1 is administered to a patient possessing one human CFTR mutation selected from selected from G178R, G551S, G970R, G1244E, S1255P, G1349D, S549N, S549R and S1251N.

11. The method of claim 9, wherein Compound 1 is administered to a patient possessing one human CFTR mutation selected from E193K, F1052V and G1069R.

12. The method of claim 4, wherein Compound 1 is administered to a patient possessing one human CFTR mutation selected from R117C, D110H, R347H, R352Q, E56K, P67L, L206W, A455E, D579G, S1235R, S945L, R1070W, F1074L, D110E, D1270N and D1152H.

13. The method of claim 5, wherein the human possesses one human CFTR mutation selected from ΔF508, R117H, and G551D.

14. The method of claim 6, wherein the human possesses one human CFTR mutation selected from ΔF508, R117H, and G551D.

15. The method of claim 7, wherein the human possesses one human CFTR mutation selected from ΔF508, R117H, and G551D.

16. The method of claim 8, wherein the human possesses one human CFTR mutation selected from ΔF508, R117H, and G551D.

17. The method of claim 1 or 4, wherein the CFTR mediated disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulinemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Pelizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebellar ataxia type I, spinal and bulbar muscular atrophy, dentatorubral pallidoluysian, and myotonic dystrophy, as well as spongiform encephalopathies, such as hereditary Creutzfeldt-Jakob disease (due to prion protein processing defect), Fabry disease, Gerstmann-Sträussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

18. The method of claim 17, wherein the CFTR mediated disease is cystic fibrosis, COPD, emphysema, dry eye disease, or osteoporosis.

19. The method of claim 18, wherein the CFTR mediated disease is cystic fibrosis.

20. The method according to claim 19, wherein the treatment includes lessening the severity of cystic fibrosis in the patient.

21. The method according to claim 19, wherein the treatment includes lessening the severity of symptoms of cystic fibrosis in the patient.

22. The method according to claim 17, wherein the patient also possesses the G551D mutation of human CFTR.

23. The method of claim 22, wherein the patient also possesses the G551D mutation of human CFTR on at least one allele.

24. The method of claim 22, wherein the patient also possesses the G551D mutation of human CFTR on both alleles.

25. The method according to claim 17, wherein the patient also possesses the ΔF508 mutation of human CFTR.

26. The method of claim 25, wherein the patient also possesses the ΔF508 mutation of human CFTR on at least one allele.

27. The method of claim 25, wherein the patient also possesses the ΔF508 mutation of human CFTR on both alleles.

28. The method according to claim 17, wherein the patient also possesses the R117H mutation of human CFTR.

29. The method of claim 28, wherein the patient also possesses the R117H mutation of human CFTR on at least one allele.

30. The method of claim 28, wherein the patient also possesses the R117H mutation of human CFTR on both alleles.
